(19) United States
(12) Patent Application Publication
(10) Pub. No.: US 2006/0024819 A1
(43) Pub. Date: Feb. 2, 2006

(54) Integration vectors

(76) Inventor: Robert E. Finney, Shoreline, WA (US)

Correspondence Address: PETER J. KNUDSEN, KNUDSEN, ATTORNEY AT LAW LLC, 13710 RIVIERA PLACE NE, SEATTLE, WA, 98125, US

(21) Appl. No.: 10/903,001

(22) Filed: Jul. 30, 2004

(51) Int. Cl. C12N 15/74

(52) U.S. Cl. 435/320.1

(57) ABSTRACT

The invention relates to integration vectors for modifying a target genomic region comprising, in a 5′ to 3′ direction, a splice acceptor site, a 3′ hybrid recognition site, and a marker sequence (i.e., a 5′ gene trap vector); or alternatively comprising, in a 5′ to 3′ direction, a marker sequence; a 5′ hybrid recognition site; and a splice donor site (i.e., a 3′ gene trap vector). The integration vector, upon insertion into the target genomic region is capable of producing a recombinant RNA transcript that is comprised of a hybrid recognition site for a selection molecule. The hybrid recognition site of recombinant RNA produced from insertion of the 5′ gene trap vector is comprised of a 5′ hybrid recognition site derived from genomic sequence and a 3′ hybrid recognition site derived from vector sequence. The hybrid recognition site of recombinant RNA produced from insertion of the 3′ gene trap vector is comprised of a 5′ hybrid recognition site derived from vector sequence and a 3′ hybrid recognition site derived from genomic sequence. The selection molecule selects recombinant cells comprising the integration vector inserted within the target genomic region.

### BACKGROUND OF THE INVENTION

Whole genome screening of RNAi, cDNA, antibody, and chemical libraries provides a viable approach to drug discovery. The availability of the human genome sequence and the results of genomic analyses (e.g., expression arrays, proteomics, and bioinformatics) create a need for cell-based assays involving specific genes of interest. Cell-based assays bridge genomic information to pathway, target, and biomarker and drug discovery. Conventional methods of utilizing genomic information require excessive time and cost, resulting in delayed marketing of new biomarkers and drug products.

Genomic information may be used to target genes by using homologous recombination to integrate marker DNA into a gene of interest in a host cell genome. However, targeted gene insertion by homologous recombination using a wide variety of approaches has met with limited success in eukaryotic cells. The limitation of this approach stems from the random integration of a vast majority of vectors instead of homologous recombination within the gene of interest. On average, the number of homologous recombinants, in the absence of direct selection or enrichment, is less than 1 in 10,000 integration events. Recombination efficiency apparently neither relates to transcriptional activity nor chromosomal location of the gene of interest. Because of the low frequencies of homologous recombinants, and the large background of random integration events that must be screened, it is impractical, if not impossible to target many genes without innovative strategies.

Special strategies have been developed to screen or select homologous recombinants from the large background of non-homologous or random integration events. When the targeted gene is itself a dominant selectable marker, homologous recombinants may be selected directly. For example, knocking out the gene encoding hypoxanthine-guanine phosphoribosyltransferase results in increased tolerance of the base analog 6-thioguanine. This particular gene-specific method is not widely applicable to most genes of interest.

Accordingly, there exists an unmet need to utilize gene trap vectors along with a gene-specific screening method to identify homologously recombined vectors that integrated into the gene of interest.

### BRIEF SUMMARY OF THE INVENTION

One of the objects of the present invention is to genetically engineer mammalian genomes by integrating specific vectors followed by screening methods that allow for selection of those cells having the vector inserted into a gene of interest. Another object of the invention is to provide a method for genetically engineering eukaryotic genomes that requires only routine laboratory procedures.

Another object of the invention is to use gene trapping to provide an effective means of modifying potentially any gene. In conventional methods, gene trap vectors generally are nonspecifically inserted into the target cell genome; the method of the invention allows for selection of the vector following integration into a gene of interest.

Another object of the invention is to integrate gene trap vectors into introns of genomic regions to allow cellular splicing machinery to produce recombinant mRNAs having both genomic and vector sequences. Another objection of the invention is to utilize gene trap vectors that contain marker sequences that are preceded by SA sequences lacking a vector promoter; such that when the vectors integrate into a gene, the cellular splicing machinery splices exons of the trapped gene onto the 5′ end of the marker sequence and, if the gene has inserted into an intron, it will be expressed, and thereby identified by selecting for cells that synthesize recombinant mRNA.

Another object of the invention is to provide methods of linking specific genomic sequences to cell-based assays, including gene expression reporter cells, gene knock-out cells, gene replacement cells, and promoter knock-in cells. Another object of the invention is to efficiently utilize genomic sequence data for pathway, target, and biomarker and drug discovery. Another object of the invention is to provide a specialized “gene-specific” technology for efficient selection of cells integrating DNA by homologous recombination so that, following selection, efficiencies of homologous to randomly integrants are substantially improved. Another object of the invention is to modify genes in a wide variety of eukaryotic cells, including: human disease-relevant cells; extensive panels of cells available from ATCC; human stem cells; mouse stem cells and embryonic stem cells; other mammalian cells (e.g., porcine, murine, bovine cells); and plants. Another object of the invention is a method that does not require vector library construction. Another object of the invention is a method that produces genetically engineered cells in less than six weeks.

Another object of the invention is to genetically modify virtually any gene of interest in the genome, including: genes whose expression is associated with a disease, disease pathways, poor clinical prognosis, or patient relapse or adverse events; and genes induced by drug products with known or unknown targets or mechanisms of action.

Another object of the invention is to develop gene-specific, cell-based assays. Another object of the invention is to generate reporter cells, in which a reporter gene (e.g., β-galactosidase or β-lactamase) is specifically placed down-stream of an endogenous promoter, such that regulation of expression of the endogenous gene is monitored by the activity of the reporter. Another object of the invention is to obtain reporter cells that track expression of specific genes during differentiation or disease progression, or in response to environmental stimuli, such as growth factors, cytokines, siRNA reagents, chemical agents, and drug products. Another object of the invention is to integrate reporters at natural chromosomal loci in which regulatory elements reside as much as 50 kb away from the promoter. Another object of the invention is a method to produce gene knockout cells to achieve stable ablation of up to 100% of gene product activity with absolute specificity and to produce knock-out cells for analysis as gene-specific disease models. Another object of the invention is to produce gene replacement cells, in which endogenous genes are replaced with alternative nucleotide sequences, such as foreign genes, mutant alleles, single nucleotide polymorphisms (SNPs), or splice variants of endogenous genes. Another object of the invention is to produce cells that are useful for examining allele specific disease models, or to enhance protein or metabolite production in cells. Another object of the invention is to produce promoter knock-in cells, in which promoters are introduced to drive expression of endogenous nucleotide sequences, including genes.

Another object of the invention is to produce unique and novel gene expression reporters. Another object of the invention is to use unique reporter vector elements to detect genes expressed at low levels, and to enhance stability of reporter activity over cell passages. Another object of the invention is to provide reporter cells in which a variety of reporters are used and exchanged, including SEAP, luciferase, GFP, β-galactosidase, and β-lactamase. Another object of the invention is to produce reporter cells that are amenable to high throughput screening.

Another object of the invention is to utilize induction cloning to produce reporter genes that are responsive to specific disease associated stimuli, such as growth factors, cytokines, and oncogenes, and to identify genes whose activity is implicated in disease-relevant signaling pathways and those associated with responses to chemical entities.

One embodiment of the invention is an integration vector for modifying a target genomic region comprising, in a 5′ to 3′ direction: a splice acceptor site (“SA”); a 3′ hybrid recognition site; and a marker sequence. Related embodiments are the integration vector further comprising a first FLP recombinase target sequence located 5′ to the marker sequence and a second FLP recombinase target sequence 3′ to the marker sequence; a polyadenylation site (“PA”) or a splice donor site (“SD”), in which either site is 3′ to the marker sequence; a bacterial origin of replication (“ORI”) or a bacterial promoter operably linked to a bacterial selection marker or both. Another related embodiment is the integration vector further comprising an internal ribosome entry site (“IRES”) 5′ to the marker sequence, preferably the 5′ leader sequence of the mRNA encoding GTX homeodomain protein. Another preferred embodiment is the integration vector in which the marker sequence consisting of a thymidine kinase, β-galacatosidase, neomycin resistance fusion gene (“TKβGEO”). Another preferred embodiment is the integration vector that lacks internal sites for cutting by a frequently cutting restriction enzyme. Another preferred embodiment is the integration vector further comprising a first homologous domain 5′ or 3′ to the marker sequence or and a first homologous domain 5′ to the marker sequence and a second homologous domain 3′ to the marker sequence, in which the first and second homologous domains have substantial homology to first and second nucleic acid sequences of the target genomic region. Another embodiment is the integration vector in which the genome region comprises a cellular gene, and the marker sequence is a splice variant of the gene, a replacement for the gene, a mutant sequence of the gene, a SNP variant of the gene, or a promoter to express the gene. Another embodiment is the integration vector further comprising one or more stop codons 5′ to the IRES. Another embodiment is the integration vector comprising, from 5′ to 3′, a SA, a 3′ hybrid recognition site, an IRES, a marker sequence, and either a PA or a SD. Another embodiment is the integration vector, further comprising a stop codon 5′ to an IRES and a bacterial promoter operably linked to a bacterial selection marker.

Another embodiment of the invention is an integration vector for modifying a genome region, comprising, in a 5′ to 3′ direction: a marker sequence; a 5′ hybrid recognition site; and a SD. Another embodiment is a 3′ integration vector further comprising a first FLP recombinase target sequence located 5′ to the marker sequence and a second FLP recombinase target sequence 3′ to the marker sequence. Another embodiment is a 3′ integration vector further comprising an ORI or a bacterial promoter operably linked to a bacterial selection marker or both. Another embodiment is this 3′ integration vector in which the marker sequence is TKβGEO. Another embodiment is the 3′ integration vector further comprising a nucleotide sequence that cannot be cut by a frequently cutting restriction enzyme. Another embodiment is the 3′ integration vector further comprising a first homologous domain or a first homologous domain 5′ to the marker sequence and a second homologous domain 3′ to the marker sequence, in which the first and second homologous domains have substantial homology with first and second nucleic acid sequences of the target genomic region. Another embodiment is the 3′ integration vector in which the genome region comprises a cellular gene, and in which the vector further comprises a nucleotide sequence selected from the group consisting of a splice variant of the gene, a replacement for the gene, a mutant sequence of the gene, a SNP variant of the gene, and a promoter to express the gene. Another embodiment is the 3′ integration vector further comprising a promoter that is operably linked to the marker sequence. Another embodiment is the 3′ integration vector further comprising, from 5′ to 3′, a promoter operably linked to the marker sequence, a 5′ hybrid recognition site, and a SD.

Another embodiment of the invention is an integration vector comprising, from 5′ to 3′, a SA, a 3′ hybrid recognition site, an IRES, a marker sequence, a 5′ hybrid recognition site, and a SD. Another embodiment is the integration vector further comprising a stop codon 5′ to the IRES and 3′ to the SA, and a bacterial promoter placed between the SA and the marker gene that is operably linked to a bacterial selection marker.

Another embodiment of the invention is a method for modifying a genomic region in a eukaryotic cell comprising the steps of: introducing an integration vector comprising a marker sequence into a population of cells; introducing into the resulting population a selection molecule comprising a nucleotide sequence homologous to RNA transcribed by the genomic region; and selecting a cell from the population in which the selection molecule inhibits expression of the marker sequence.

Another embodiment of the invention is a method for modifying a genomic region in a eukaryotic cell comprising the steps of: introducing into a cell an integration vector comprising, in a 5′ to 3′ direction, a marker sequence, a 5′ hybrid recognition site, and a SD; introducing into the resulting population a selection molecule comprising a first nucleotide sequence homologous to the 5′ hybrid recognition site and a second nucleotide sequence homologous to RNA transcribed by the genomic region; and selecting a cell from the population in which the selection molecule inhibits expression of the marker sequence.

Another embodiment of the invention is a method for modifying a genomic region in a eukaryotic cell comprising the steps of: introducing into a cell an integration vector comprising, in a 5′ to 3′ direction, a SA, a 3′ hybrid recognition site, and a marker sequence; introducing into the resulting population a selection molecule comprising a first nucleotide sequence homologous to RNA transcribed by the genomic region and a second nucleotide sequence homologous to the 3′ hybrid recognition site; and selecting a cell from the population in which the selection molecule inhibits expression of the marker sequence.

Another embodiment of the invention is a method for modifying a gene in a eukaryotic cell comprising the steps of: introducing into a population of cells an integration vector comprising a promoter operably linked to a marker sequence, a first FLP recombinase site and a second FLP recombinase site; introducing into the resulting population a selection molecule comprising a nucleotide sequence homologous to a RNA transcribed by the genomic region; selecting a cell from the population in which the selection molecule inhibits expression of the marker sequence; incubating the selected cells with a second vector comprising a first FLP recombinase site and a second FLP recombinase site, and the FLP recombinase, and selecting cells in which the promoter, the marker sequence or both are removed.

Another embodiment of the invention is a method for modifying a gene in a eukaryotic cell comprising the steps of: introducing into a population of cells an integration vector comprising a first marker sequence, a first FLP recombinase site 5′ to the first marker sequence and a second FLP recombinase site 3′ to the first marker sequence; introducing into the resulting population a selection molecule comprising a nucleotide sequence homologous to RNA transcribed by the genomic region; selecting a cell from the population in which the selection molecule inhibits expression of the first marker sequence; incubating the selected cells with a conversion vector comprising a second marker sequence, a first FLP recombinase site 5′ to the second marker sequence and a second FLP recombinase site 3′ to the second marker sequence, and the FLP recombinase, in which the second marker sequence is selected from the group consisting of a splice variant of the gene, a replacement for the gene, a mutant sequence of the gene, a SNP variant of the gene, and a promoter to express the gene; and selecting cells in which the first marker sequence is replaced by the second marker sequence.

Another embodiment of the invention is the method of the previous paragraph, in which the integration vector further comprises a promoter operably linked to the first marker sequence. Another embodiment is the method in which the integration vector further comprises a SA contiguous with a 3′ hybrid recognition site, and in which the selection molecule comprises a nucleotide sequence having homology to the hybrid recognition site. Another embodiment is the method in which the integration vector further comprises a 5′ hybrid recognition site contiguous with a SA, and in which the selection molecule comprises a nucleotide sequence having homology to the hybrid recognition site. Another embodiment is the method in which the region of the gene of interest is an exon, an intron, a 5′UTR or a 3′UTR of the gene. Another embodiment is the method in which the integration vector further comprises a first homologous domain, or a first homologous domain 5′ to the marker sequence and a second homologous domain 3′ to the marker sequence, in which the first and second homologous domains have substantial homology with first and second nucleic acid sequences of the target genomic region. Another embodiment is the method in which the genome region comprises a cellular gene, and in which the integration vector further comprises a nucleotide sequence selected from the group consisting of a splice variant of the gene, a replacement for the gene, a mutant sequence of the gene, a SNP variant of the gene, and a promoter to express the gene. Another embodiment is the method in which the integration vector further comprises a PA or a SD, in which either site is 3′ to the marker sequence. Another embodiment is the method in which the integration vector further comprises a ORI or a bacterial promoter operably linked to a bacterial selection marker or both. Another embodiment is the method in which the integration vector further comprises an IRES 5′ to the marker sequence, preferably comprising a 5′ leader sequence of the mRNA encoding GTX homeodomain protein. Another preferred embodiment is the method in which the marker sequence is TKβGEO. Another embodiment is the method in which the integration vector lacks internal sites for cutting by a frequently cutting restriction enzyme. Another embodiment is the method in which the integration vector further comprises one or more stop codons 5′ to the IRES. Another embodiment is the method in which the integration vector further comprises, from 5′ to 3′, a SA, a 3′ hybrid recognition site, an IRES, a marker sequence, and either a PA or a SD.

Another embodiment of the invention is an mRNA molecule comprising a nucleic acid sequence having sequence encoded by a gene of interest, a recognition site, and a marker sequence. Another embodiment is the mRNA molecule in which the recognition site comprises a nucleic acid sequence having homology to the gene of interest. Another embodiment is the mRNA molecule further comprising an IRES 5′ to the marker sequence, preferably comprising a sequence encoding GTX homeodomain protein. Another embodiment is the mRNA molecule in which the marker sequence is TKβGEO.

Another embodiment of the invention is an mRNA molecule derived from an integration vector, the vector comprising a 3′ hybrid recognition site 5′ to a marker sequence. Another embodiment is the mRNA molecule derived from an integration vector, the vector comprising a marker sequence and a 5′ hybrid recognition site.

Another embodiment of the invention is a cell having a vector integrated within a genomic region, in which the vector comprises, in a 5′ to 3′ direction: a SA; a 3′ hybrid recognition site; and a marker sequence. Another embodiment is the cell in which the vector further comprises a first FLP recombinase target sequence located 5′ to the marker sequence and a second FLP recombinase target sequence 3′ to the marker sequence. Another embodiment is the cell in which the vector further comprises a PA or a SD, in which either site is 3′ to the marker sequence. Another embodiment is the cell in which the vector further comprises a ORI or a bacterial promoter operably linked to a bacterial selection marker or both. Another embodiment is the cell in which the vector further comprises an IRES between the recognition site for a selection molecule and the marker sequence, preferably a 5′ leader sequence of the mRNA encoding GTX homeodomain protein. Another embodiment is the cell in which the marker sequence is a TKβGEO. Another embodiment is the cell in which the vector lacks internal sites for cutting by a restriction enzyme. Another embodiment is the cell in which the vector further comprises a first homologous domain or a first homologous domain 5′ to the marker sequence and a second homologous domain 3′ to the marker sequence, in which the first and second homologous domains have substantial homology with first and second nucleic acid sequences of the target genomic region. Another embodiment is the cell in which the genomic region comprises a cellular gene, and in which the vector further comprises a nucleotide sequence selected from the group consisting of a splice variant of the gene, a replacement for the gene, a mutant sequence of the gene, a SNP variant of the gene, and a promoter to express the gene. Another embodiment is the cell in which the vector further comprises one or more stop codons 5′ to the IRES. Another embodiment of the invention is a cell in which the vector further comprises, from 5′ to 3′, a SA, a 3′ hybrid recognition site, an IRES, a marker sequence, and either a PA or a SD. Another embodiment is the cell in which the vector further comprises a stop codon 5′ to an IRES and a bacterial promoter operably linked to a bacterial selection marker.

Another embodiment of the invention is a cell having a vector integrated within a genomic region, in which the vector comprises: a marker sequence; a 5′ hybrid recognition site; and a SD. Another embodiment is the cell in which the vector further comprises a first FLP recombinase target sequence located 5′ to the marker sequence and a second FLP recombinase target sequence 3′ to the marker sequence. Another embodiment is the cell in which the vector further comprises a ORI or a bacterial promoter operably linked to a bacterial selection marker or both. Another embodiment is the cell in which the marker sequence is a TKβGEO. Another embodiment is the cell in which the vector lacks internal sites for cutting by a restriction enzyme. Another embodiment is the cell in which the vector further comprises a first homologous domain or a first homologous domain 5′ to the marker sequence and a second homologous domain 3′ to the marker sequence, in which the first and second homologous domains have substantial homology with first and second nucleic acid sequences of the target genomic region. Another embodiment is the cell in which the genome region comprises a cellular gene, and in which the vector further comprises a nucleotide sequence selected from the group consisting of a splice variant of the gene, a replacement for the gene, a mutant sequence of the gene, a SNP variant of the gene, and a promoter to express the gene. Another embodiment is the cell in which the vector further comprises a promoter that is operably linked to the marker sequence. Another embodiment is the cell in which the vector further comprises, from 5′ to 3′, a promoter operably linked to the marker sequence, a 5′ hybrid recognition site, and a splice donor.

Another embodiment of the invention is a cell having a vector integrated within a genomic region, in which the vector comprises, a 3′ hybrid recognition site, an IRES, a marker sequence, a 5′ hybrid recognition site, and a splice donor. Another embodiment is the cell in which the vector further comprises a stop codon 5′ to the IRES and 3′ to the SA, and a bacterial promoter placed between the SA and the marker gene that is operably linked to a bacterial selection marker.

Another embodiment of the invention is an eukaryotic cell having a modified genomic region, in which the cell is a product of a process comprising the steps of: introducing an integration vector comprising a marker sequence into a population of cells; introducing into the resulting population a selection molecule comprising a nucleotide sequence having homology to RNA transcribed by the genomic region; and selecting a cell from the population in which the selection molecule inhibits expression of the marker sequence.

Another embodiment of the invention is an eukaryotic cell having a modified genomic region, in which the cell is a product of a process comprising the steps of: introducing into a cell an integration vector comprising, in a 5′ to 3′ direction, a marker sequence, a 5′ hybrid recognition site, and a SD; introducing into the resulting population a selection molecule comprising a first nucleotide sequence having homology to the 5′ hybrid recognition site and a second nucleotide sequence having homology to RNA transcribed by the genomic region; and selecting a cell from the population in which the selection molecule inhibits expression of the marker sequence.

Another embodiment of the invention is an eukaryotic cell having a modified genomic region, in which the cell is a product of a process comprising the steps of: introducing into a cell an integration vector comprising, in a 5′ to 3′ direction, a SA, a 3′ hybrid recognition site, and a marker sequence; introducing into the resulting population a selection molecule comprising a first nucleotide sequence having homology to RNA transcribed by the genomic region and a second nucleotide sequence having homology to the 3′ hybrid recognition site; and selecting a cell from the population in which the selection molecule inhibits expression of the marker sequence.

Another embodiment of the invention is an eukaryotic cell having a modified gene, in which the cell is a product of a process comprising the steps of: introducing into a population of cells an integration vector comprising a promoter operably linked to a marker sequence, a first FLP recombinase site and a second FLP recombinase site; introducing into the resulting population a selection molecule comprising a nucleotide sequence having homology to a RNA transcribed by the genomic region; selecting a cell from the population in which the selection molecule inhibits expression of the marker sequence; incubating the selected cells with a second vector comprising a first FLP recombinase site and a second FLP recombinase site, and the FLP recombinase, and selecting cells in which the promoter, the marker sequence or both are removed. Another embodiment is the cell in which the integration vector further comprises a promoter operably linked to the first marker sequence. Another embodiment is the cell in which the integration vector further comprises a SA contiguous with a 3′ hybrid recognition site, and in which the selection molecule comprises a nucleotide sequence having homology to the hybrid recognition site. Another embodiment is the cell in which the integration vector further comprises a 5′ hybrid recognition site contiguous with a SD, and in which the selection molecule comprises a nucleotide sequence having homology to the hybrid recognition site.

Another embodiment of the invention is an eukaryotic cell having a modified genomic region, in which the cell is a product of a process comprising the steps of: introducing into a population of cells an integration vector comprising a first marker sequence, a first FLP recombinase site 5′ to the first marker sequence and a second FLP recombinase site 3′ to the first marker sequence; introducing into the resulting population a selection molecule comprising a nucleotide sequence having homology to RNA transcribed by the genomic region; selecting a cell from the population in which the selection molecule inhibits expression of the first marker sequence; incubating the selected cells with a conversion vector comprising a second marker sequence, a first FLP recombinase site 5′ to the second marker sequence and a second FLP recombinase site 3′ to the second marker sequence, and the FLP recombinase, in which the second marker sequence is selected from the group consisting of a splice variant of the gene, a replacement for the gene, a mutant sequence of the gene, a SNP variant of the gene, and a promoter to express the gene; and selecting cells in which the first marker sequence is replaced by the second marker sequence.

Another embodiment of the invention is an eukaryotic cell having a modified genomic region, produced by any one of the processes, infra, in which genomic region is an exon, an intron, a 5′UTR, or a 3′UTR of a gene. Another embodiment is the cell in which the integration vector further comprises a first homologous domain or a first homologous domain 5′ to the marker sequence and a second homologous domain 3′ to the marker sequence, in which the first and second homologous domains have homology with first and second nucleic acid sequences of the target genomic region. Another embodiment is the cell in which the genome region comprises a cellular gene, and in which the integration vector further comprises a nucleotide sequence selected from the group consisting of a splice variant of the gene, a replacement for the gene, a mutant sequence of the gene, a SNP variant of the gene, and a promoter to express the gene. Another embodiment is the cell in which the integration vector further comprises a PA or a SD, in which either site is 3′ to the marker sequence. Another embodiment is the cell in which the integration vector further comprises a ORI or a bacterial promoter operably linked to a bacterial selection marker or both. Another embodiment is the cell in which the integration vector further comprises an IRES 5′ to the marker sequence, preferably the 5′ leader sequence of the mRNA encoding GTX homeodomain protein. Another embodiment is the cell in which the marker sequence is a TKβGEO. Another embodiment is the cell in which the integration vector lacks internal sites for cutting by a frequently cutting restriction enzyme. Another embodiment is the cell in which the integration vector further comprises one or more stop codons 5′ to the IRES. Another embodiment is the cell in which the the integration vector further comprises, from 5′ to 3′, a SA, a 3′ hybrid recognition site, an IRES, a marker sequence, and either a PA or a SD.

Another embodiment of the invention is an eukaryotic cell comprising a mRNA molecule in which the mRNA comprises a sequence encoded by a gene of interest, a recognition site, and a marker sequence. Another embodiment is the cell in which the recognition site comprises a nucleic acid sequence having homology to the gene of interest. Another embodiment is the cell in which the mRNA further comprises an IRES 5′ to the marker sequence, preferably the 5′ leader sequence of the mRNA encoding GTX homeodomain protein. Another embodiment is the cell in which the marker sequence is a TKβGEO. Another embodiment is the cell in which the RNA molecule is derived from an integration vector, and the vector comprising a 3′ hybrid recognition site 5′ to a marker sequence. Another embodiment is the cell in which the RNA molecule is derived from an integration vector, the vector comprising a marker sequence 5′ to a 5′ hybrid recognition site.

### DETAILED DESCRIPTION OF THE INVENTION

Stable integration of marker DNA into a cellular genome results from two separate mechanisms: random integration or homologous recombination. During random integration, introduced DNA integrates randomly at a large number of potential locations throughout the genome. During homologous recombination, introduced DNA interacts with and integrates into a site in the genome that contains a substantially homologous DNA sequence, usually within a gene of interest. In higher eukaryotic cells the frequency of homologous recombination is far less than the frequency of random integration. The ratio of these frequencies has direct implications for gene targeting, in which integration occurs through homologous recombination.

Gene targeting represents a major advance in the ability to selectively manipulate an eukaryotic cell genome. Using this technique, a particular DNA sequence can be targeted and modified in a site-specific and precise manner. Different types of DNA sequences can be targeted for modification, including regulatory regions, nontranscribed regions, transcribed regions, untranslated regions, coding regions and introns. By modifying regulatory regions, e.g., promoter regions, terminator regions and enhancer regions, the timing and level of expression of a gene can be altered. Coding regions can be modified, for example, to alter, enhance or eliminate the activity of an enzyme, the sensitivity of protein to inactivation, or to initiate apoptosis. Introns and exons are suitable targets for modification. Modifications of DNA sequences can involve insertions, deletions, or substitutions. One example of a modification is inactivation of a gene by site-specific integration of a nucleotide sequence that disrupts expression of the gene product to result in “knock out” of the gene by targeting.

One aspect of the present invention is a method of modifying the genome of eukaryotic cells, preferably mammalian cells, most preferably human and mouse cells, to produce a recombinant cell. The method of the invention comprises steps of integrating a marker sequence into a genetic locus of interest and selecting recombinant cells, in which expression of the marker sequence is changed by selection molecules, such as antisense molecules, ribozymes, and RNA interference (RNAi) molecules. In a preferred method, the selection molecules interact with a recognition site that is substantially specific to the RNA product of the genetic locus of interest. Another method of the invention comprises steps of integrating a vector of the invention into a genetic locus of interest in which a substantially unique marker is created as a result of integration of the vector. In a preferred method, the substantially unique marker is comprised of RNA sequence derived from the genetic locus of interest combined with RNA sequence derived from the vector. In another preferred method, the substantially unique marker is comprised of a polypeptide fragment derived from the genetic locus of interest combined with a polypeptide fragment derived from the vector. A preferred method includes selecting recombinant cells in which expression of the created marker sequence is changed by selection molecules, such as antisense molecules, ribozymes, and RNA interference (RNAi) molecules. Another preferred method includes selecting recombinant cells using analytical selection molecules, such as antibodies directed at the marker and analytical probes sharing sequence homology with the marker to detect cells expressing the marker. In a preferred embodiment, the genetic locus is a gene. In another preferred embodiment, the method of the invention includes an additional step of selecting recombinant cells for their expression of the marker sequence.

Transcription of eukaryotic genomes occurs at many loci within the genome and serves multiple functions. On the one hand, transcription serves to express RNA that does not encode for polypeptides. Transcripts of pseudogenes and transcripts derived from retroviral elements residing in the genome may not provide for polypeptide synthesis. On the other hand, transcription serves to express RNA for genes that encode for polypeptides.

Genes may consist of a promoter and a single exon in which termination occurs at the end of the exon, typically at a PA. These intronless genes may contain a 5′ untranslated region, a translated region, and a 3′ untranslated region. Most genes consist generally of a promoter and one or more exons with intervening intron sequences. Primary transcripts of genes typically include both exons and introns and terminate at PAs. The first exon of a multiple-exon gene contains a SD at its 3′ end, while the last exon of a gene contains a SA at its 5′ end and a PA at its 3′ end. Exons between the first and last exon contain both a SA site at the 5′ end and a splice donor at the 3′ end. The primary transcript is processed by cellular splicing machinery to generate mRNA, comprised of exons that terminate at the PA. These mRNAs may contain 5′ untranslated regions, translated regions that typically start at an ATG “start” codon and terminate at a termination codon, and 3′ untranslated regions that reside 3′ to the translation termination codon.

In one embodiment of the invention, the marker sequence is present in a targeting vector comprising the marker sequence flanked by DNA homologous to a region within the genetic locus of interest. Upon introduction into a cell, the targeting vector integrates into the genetic locus of interest by homologous recombination. A recombinant genetic locus is produced by integration of the integration vector into the genetic locus of interest. In one embodiment of the present invention, integration is into non-trasncribed DNA. In another embodiment, integration is into transcribed DNA. In another embodiment, integration is into a gene. In another embodiment, integration is into an exon of a gene. In another embodiment, integration is into the 5′ untranslated region of a gene. In another embodiment, integration is into the translated region of a gene. In another embodiment, integration is into the 3′ untranslated region of a gene. In a preferred embodiment, integration is into an intron of a gene.

The recombinant genetic locus expresses a recombinant RNA molecule that comprises nucleic acid sequence corresponding to a region of genomic DNA and a region of the marker sequence. In another embodiment of the invention, recombinant cells with the recombinant genetic locus are selected by inducing or introducing RNAi molecules that comprise nucleic acid sequence corresponding to the region of the genetic locus transcribed in the recombinant mRNA molecule. In a preferred embodiment, recombinant cells are first selected for expression of the marker sequence, and then selected for reduced expression of the marker sequence.

The Integration Vector

Integration vectors of the invention include all vectors capable of inserting at least a portion of their polynucleotide sequence into a genomic regions, including genes, and upon insertion, are capable of producing recombinant RNA molecules. The invention includes vectors known in the art that may be used to transfer an exogenous DNA sequence into the genome of a cell. Vectors of the invention may be plasmids, viruses, or retroviruses, and the like, for example. Specific embodiments of the invention include gene trap vectors and targeting vectors. Methods of designing and constructing vectors, and methods of introducing vector sequences into a genome are well known in the art and are described, for example, in GENE TARGETING: A PRACTICAL APPROACH, 2nd ed. (2000), Joyner, A. L., ed., Oxford University Press, New York; GENE TARGETING PROTOCOLS (METHODS IN MOLECULAR BIOLOGY, VOL. 133), (2000), Kmiec, E. B. and Gruenert, D. C., eds., Humana Press; and Torres, R. M. et al., LABORATORY PROTOCOLS FOR CONDITIONAL GENE TARGETING (1997), Oxford University Press, Oxford; and references cited within, all of which are incorporated by reference. Specific vectors are also described in U.S. Pat. No. 5,364,783, No. 5,464,764 (positive-negative selection), No. 5,487,992, No. 5,627,059, No. 5,631,153, No. 5,719,055 (transposons), U.S. Pat. No. 5,830,698, No. 5,998,144, No. 6,280,937, No. 6,284,541, Nos. 6,139,833, 6,303,327, No. 6,319,692, No. 6,329,200, and No. 6,080,576, and references and patents cited therein. Generally, vectors of the invention may be constructed, propagated, isolated, and examined using routine molecular biology techniques such as restriction enzyme digestion, polymerase chain reaction, ligation, transformation, and southern blotting, according to procedures well known in the art and described in CURRENT PROTOCOLS IN MOLECULAR BIOLOGY, (2001), Ausubel et al. (eds.), John Wiley & Sons, New York and Sambrook, et al., MOLECULAR CLONING: A LABORATORY MANUAL, (2001), Cold Spring Harbor Laboratory Press, Cold Spring Harbor, N.Y., and U.S. Pat. No. 5,789,215, for example. Alternatively, vectors may be synthesized (e.g., as described by U.S. Pat. No. 6,664,112).

Generally, gene trap vectors randomly integrate within genes. Gene trap vectors are described, for example, in U.S. Pat. No. 6,218,123, No. 6,207,371, No. 6,139,833, and No. 6,080,576, and references cited within, all of which are hereby incorporated by reference in their entirety. Examples of gene trap vectors contemplated by the invention include promoter trap vectors, exon trap vectors, and 3′ polyadenylation (PA) trap vectors. In addition, gene trap vectors contemplated by the invention include secretion trap vectors and conventional gene trap vectors designed for insertion into an intron.

The integration vector of the invention functions not only to trap genes, but also to trap other transcribed or nontranscribed genomic sequences. The integration vector of the invention further functions to introduce a marker sequence into genomic DNA, in which recombinant RNA is produced. The recombinant RNA is comprised of sequences derived from the genome and sequences derived from vector DNA. The RNA may be a primary RNA transcript, or a processed RNA transcript, including mRNA. The processed RNA may encode a polypeptide, or it may contain marker sequence that is independent of translation. The composition of the integration vector differs according to the site of integration within the targeted genomic site of interest. A vector that results in a RNA comprised of sequences derived from the genome and sequences derived from vector DNA may be used for the invention. Examples of integration vectors meeting these criteria are provided herein for integration into transcribed DNA, for integration into nontranscribed DNA, for integration into 5′ untranslated regions of exons, for integration into translated regions of exons, for integration into introns, and for integration into 3′ untranslated regions of exons.

Integration into Nontranscribed DNA: For integration into nontranscribed DNA, the preferred vector of the invention comprises a vector promoter and a marker sequence, either alone or in combination with a SD 3′ to the marker sequence. A preferred vector of the invention further comprises an IRES 5′ to the marker sequence. If expression of the marker is mediated by a vector promoter either alone or in combination with a SD site, the recombinant RNA will include sequences derived from the marker plus sequences derived from genomic DNA residing 3′ to the integration site, in which a genomic termination signal such as a PA site may be captured.

Integration Into Transcribed DNA: For integration into transcribed DNA, the preferred vector of the invention comprises a marker sequence, either alone or in combination with one or more of the following: a PA 3′ to the marker sequence, a SD 3′ to the marker sequence, and a SA 5′ to the marker sequence. A preferred vector of the invention further comprises an IRES 5′ to the marker sequence. If a vector PA site is not used, the recombinant RNA will include sequences derived from genomic DNA that resides 5′ to the integration site and 3′ to the promoter that drives expression of the transcript, plus sequences derived from the IRES (if included) and marker, plus sequences derived from genomic DNA residing 3′ to the integration site, in which a genomic termination signal such as a PA site may be captured. If a vector PA site is used, the recombinant RNA will include sequences derived from genomic DNA that resides 5′ to the integration site and 3′ to the promoter that drives expression of the transcript plus sequences derived from the IRES (if included) and marker.

Instead of relying on an endogenous promoter for expression, a vector promoter 5′ to the marker sequence can also be used either alone or in combination with a SD site 3′ to the marker sequence. If expression is mediated by a vector promoter either alone or in combination with a SD site, the recombinant RNA will include sequences derived from the marker plus sequences derived from genomic DNA residing 3′ to the integration site, in which a genomic termination signal such as a PA site may be captured.

Integration Into 5′ Untranslated Regions of Exon DNA: For integration into 5′ untranslated regions of exon DNA, a preferred vector of the invention comprises the marker sequence either alone or in combination with a PA 3′ to the marker sequence or a SD site 3′ to the marker sequence. A preferred vector further comprises an IRES, 5′ to the marker gene. If a vector SD site is used, the recombinant RNA will include exon sequences derived from untranslated genomic DNA that resides 5′ to the integration site and 3′ to the promoter that drives expression of the transcript, plus sequences derived from the IRES (if included) and marker, plus exon sequences derived from genomic DNA residing 3′ to the integration site, in which a genomic termination signal such as a PA site may be captured. If a vector PA site is used, the recombinant RNA will include sequences derived from untranslated genomic DNA that resides 5′ to the integration site and 3′ to the promoter that drives expression of the transcript plus exon sequences derived from the IRES (if included) and the marker.

Instead of relying on an endogenous promoter for expression, a vector promoter 5′ to the marker sequence can also be used either alone or in combination with a SD site 3′ to the marker sequence. If expression is mediated by a vector promoter either alone or in combination with a SD site, the RNA will include sequences derived from the IRES (if included) and marker plus sequences derived from genomic DNA residing 3′ to the integration site, in which a genomic termination signal such as a PA site may be captured.

Integration Into Translated Regions of Exons: For integration into translated (coding) DNA, a preferred vector of the invention comprises the marker sequence, either alone or in combination with either a PA 3′ to the marker or a SD 3′ to the marker. A preferred vector further comprises an IRES residing 5′ to the marker sequence. If a vector PA site is not used, the recombinant RNA will include sequences derived from genomic DNA that reside 5′ to the integration site and 3′ to the promoter that drives expression of the transcript, sequences derived from the IRES (if included) and marker, plus sequences derived from genomic DNA residing 3′ to the integration site, in which a genomic termination signal such as a PA site may be captured. If a vector PA site is used, the recombinant RNA will include sequences derived from genomic DNA that resides 5′ to the integration site and 3′ to the promoter that drives expression of the transcript plus sequences derived from the IRES (if included) and marker.

If the marker sequence encodes a polypeptide whose reading frame is essential to its function as a marker, and if an IRES is not utilized in the vector, a functional polypeptide is obtained only if integrated in-frame with coding sequences derived from exon sequences residing 5′ to the integration site. If the marker sequence encodes a polypeptide whose reading frame is essential to its function as a marker, and if an IRES is included in the vector, the polypeptide need not be in-frame with sequences derived from exons.

Instead of relying on an endogenous promoter for expression, a vector promoter 5′ to the marker sequence can also be used either alone or in combination with a SD site 3′ to the marker sequence. If expression is mediated by a vector promoter either alone or in combination with a SD site, the recombinant RNA will include sequences derived from the IRES (if included) and marker plus sequences derived from genomic DNA residing 3′ to the integration site, in which a genomic termination signal such as a PA site may be captured.

Integration Into Introns: For integration into an intron, a preferred vector of the invention comprises a marker sequence and a PA. Another preferred vector further comprises an IRES residing 5′ to the marker. When integrated into an intron, the recombinant RNA will include sequences derived from genomic DNA that resides 5′ to the integration site and 3′ to the promoter that drives expression of the transcript plus sequences derived from the IRES (if included) and marker. If the marker sequence encodes a polypeptide whose reading frame is essential to its function as a marker, and if an IRES is not utilized in the vector, a functional polypeptide is obtained if integrated in-frame with coding sequences derived from 5′ exons and non-spliced intron sequences residing between the integration site and the exon immediately 5′ to the integration site. Alternatively, a functional polypeptide is obtained if integrated into an intron 5′ to the translation start site of a polypeptide derived from the genomic locus. If the marker sequence encodes a polypeptide whose reading frame is essential to its function as a marker, and if an IRES is included in the vector, the polypeptide need not be in-frame with sequences derived from exons and introns and it need not reside 5′ to the translation start site for the genetic locus.

Another preferred vector of the invention comprises a marker sequence and a SA that resides 5′ to the marker, either alone or in combination with a PA site that resides 3′ to the marker or a SD that resides 3′ to the marker. Another preferred vector further comprises an IRES residing 5′ to the marker. If used alone or in combination with a SD site, the recombinant RNA will include exon sequences derived from genomic DNA that resides 5′ to the integration site and 3′ to the promoter that drives expression of the transcript, sequences derived from the IRES (if included) and marker, plus sequences derived from genomic DNA residing 3′ to the integration site, in which a genomic termination signal such as a PA site may be captured. If a vector PA site is used, the recombinant RNA will include exon sequences derived from genomic DNA that resides 5′ to the integration site and 3′ to the promoter that drives expression of the transcript plus sequences derived from the IRES (if included) and marker.

If the marker sequence encodes a polypeptide whose reading frame is essential to its function as a marker, and if an IRES is not utilized in the vector, a functional polypeptide is obtained if integrated in-frame with coding sequences derived from exon sequences residing 5′ to the integration site. Alternatively, a functional polypeptide is obtained if integrated into an intron that resides 5′ to the translation start site of a polypeptide derived from the genomic locus. If the marker sequence encodes a polypeptide whose reading frame is essential to its function as a marker, and if an IRES is included in the vector, the polypeptide need not be in-frame with sequences derived from 5′ exons and it need not reside 5′ to the translation start site for the genetic locus.

Instead of relying on an endogenous promoter for expression, a vector promoter 5′ to the marker sequence can also be used either alone or in combination with a SD site 3′ to the marker sequence. If expression is mediated by a vector promoter either alone or in combination with a SD site, the recombinant RNA will include sequences derived from the IRES (if included) and marker plus sequences derived from genomic DNA residing 3′ to the integration site, in which a genomic termination signal such as a PA site may be captured.

Integration Into 3′ Untranslated Regions of Exon DNA: For integration into a 3′ untranslated region of an exon, the preferred vector of the invention comprises a marker sequence either alone or in combination with a PA 3′ to the marker or a SD 3′ to the marker. Another preferred vector further comprises an IRES residing 5′ to the marker. If used alone or in combination with a SD, the recombinant RNA will include exon sequences derived from genomic DNA that resides 5′ to the integration site and 3′ to the promoter that drives expression of the transcript, plus sequences derived from the IRES (if included) and marker, plus exon sequences derived from DNA residing 3′ to the integration site, in which a genomic termination signal such as a PA site may be captured. If a vector PA site is used, the recombinant RNA will include exon sequences derived from genomic DNA that resides 5′ to the integration site and 3′ to the promoter that drives expression of the transcript plus sequences derived from the IRES (if included) and marker. If the marker sequence encodes a polypeptide, inclusion of an IRES results in a bicistronic recombinant RNA; a full-length, target gene polypeptide and a marker polypeptide are both translation products.

Instead of relying on an endogenous promoter for expression, a vector promoter 5′ to the marker sequence can also be used either alone or in combination with a SD site 3′ to the marker sequence. If expression is mediated by a vector promoter either alone or in combination with a SD site, the recombinant RNA will include sequences derived from the IRES (if included) and marker plus exon sequences derived from genomic DNA residing 3′ to the integration site, in which a genomic termination signal such as a PA site may be captured.

Integration vectors of the invention may further comprise one or more recombinase recognition sites. The location of the recombinase recognition sites depends on the utility. Recombinase sites are included for recombinase-mediated deletion, inversion, insertion, or replacement of vector sequences. In a preferred vector, two recombinase recognition sites flank the marker sequence, one located 5′ of the marker sequence and the second located 3′ of the marker sequence. In another preferred vector of the invention, the recombinase recognition sites are positioned to direct recombinase-mediated deletion of the marker sequence following identification or selection of desired integration events. In another preferred vector of the invention, recombinase sites are positioned to replace the entire integration vector with alternative DNA sequences.

Integration vectors of the invention may further comprise one or more contributing vector sequences to selection molecule recognition sites. Selection molecule recognition sites may include RNA sequences derived wholly from genomic DNA sequence, or partly from genomic DNA and partly from vector DNA sequences. Selection molecule recognition sites may also encode for polypeptide sequences, in which part of the polypeptide sequence is derived from genomic DNA and part from vector DNA. Preferred selection molecules that recognize either the RNA sequence or the polypeptide sequence includes antisense molecules, ribozymes, RNAi molecules, analytical probes sharing sequence homology with the marker, and antibodies. Selection molecule recognition sites are used to select for or identify cells with vector DNA integrated into genetic loci of interest.

Preferred vectors of the invention further comprise any additional components desired to integrate into the genetic locus of interest. A preferred vector includes regulators of gene expression, such as translation stop sequences and enhancers of gene expression. Another preferred vector includes genes or coding sequences of genes. Another preferred vector includes additional exons of genes, such as those representing alternative splice variants of genes. Another preferred vector includes mutant genes or exons of genes. A preferred vector includes an alternative splice variant, SNP, or other mutant version of an exon for the gene into which the insertion vector has integrated.

In another preferred embodiment of the invention, the DNA sequence of the vector will lack restriction sites for restriction endonuclease sites that cut genomic DNA frequently. In preferred embodiments of the invention, restriction endonucleases include those that cut the host cell genome at a frequency of every 1,500 bases or less, more preferably every 1,000 bases or less, and most preferably every 100-500 bases.

Although any frequently cutting restriction endonuclease may be used for embodiments of the invention, frequently cutting restriction endonucleases typically include those whose restriction sites recognize 4 base pair (bp) palindromes (4 cutters) or 6 bp palindromes (6 cutters) with degeneracy at the 1^{st} and 6^{th} base pairs of the restriction site. Some examples of 4 cutters include Sau3A which recognizes ˆGATC, BfaI which recognizes ˆCTAG, and MspI that recognizes CˆCGG. Examples of 6 cutters with degenerated nucleotides at the ends may include EaeI that recognizes YˆGGCCR, BstYI that recognizes RˆGATCY, ApoI that recognizes RˆAATTY, and HaeII that recognizes RGCGCˆY. Lists of restriction endonucleases, including 4 cutters and 6 cutters and frequencies for cutting various organisms, including viruses, may be found at http://rebase.neb.com.

Modifying the DNA sequence by removal of restriction endonuclease restriction sites that are found frequenctly in genomes of cells makes it easier to identify sites of vector integration by inverse PCR methods. The methods typically include restriction digestion of genomic DNA, ligation under conditions that favor intramolecular (self-ligation) as opposed to concatamer formation, inverse PCR of trapped genomic sequences using sets of PCR primers complementary to sequences derived from the vector, sequencing of the PCR product, including sequences derived from trapped genomic DNA, and alignment with human genome data bases for determining gene locus, gene, and specific integration sites. By removing selected restriction endonuclease sites (e.g. 4 cutters and degenerated 6 cutters from vector sequences, inverse PCR produces will produce fragments whose sizes are more consistent than when using restriction endonucleases that cut less frequently. Inverse PCR is accordingly a more reproducible process that is amenable to multi-well cell culture plates and robotic automation.

In another embodiment of the invention, the DNA sequence of the vector will be modified to remove methylation sites and thereby to increase its stability, such as during passages involved in cell culture. Only DNA sequences that can be modified without loss of function of the marker or other vector components are modified. Methylation sites are readily identified (Shiraishi et al., Biol. Chem. 2002 383:893-906).

In another embodiment of the invention, the DNA sequence of the vector is chosen to minimize hairpin domains. For example, as opposed to an alternative IRES, the GTX sequence lacks extensive hairpin RNA structure that could impair RNAi-initiated mRNA degradation.

In another embodiment of the invention, the vector of the invention is flanked at least on one side by sequences (i.e., a homology domain) that are substantially homologous to the desired integration site for the vector. In a preferred vector, substantially homologous sequences reside both 5′ to vector sequences and 3′ to vector sequences. Vectors flanked by sequences that are substantially homologous to the desired integration site are targeting vectors for homologous recombination.

Marker Sequences and Markers

A marker sequence includes a selectable marker sequence or a reporter marker sequence. A selectable marker sequence encodes a selectable marker, and a reporter marker sequence encodes a reporter. A marker sequence encodes a selectable marker or a reporter. In a variety of embodiments, vectors, cells and animals of the invention comprise a marker sequence. For example, in one embodiment, cells comprising a vector integrated into the genome of a cell will also contain a marker sequence. Marker sequences typically comprise vectors of the invention or are created as a result of integration of vector into the genome of a cell. Markers have multiple functions, including use in methods to identify or select for a cell that has integrated vector sequences into its genome or into a desired genetic locus in its genome.

Selectable marker sequence may be positive selection markers or negative selection markers. A reporter marker sequence encodes a reporter marker including polynucleotides and polypeptides, expression of which in a cell produces a detectable signal, such as luminescence, for example. Marker sequences include genes and transcription units. In certain embodiments, a vector of the invention may contain a negative selection marker, such as those disclosed in U.S. Pat. No. 5,464,764 and No. 5,625,048, hereby incorporated by reference in its entirety. Negative selection methods typically involve removing cells that express the negative selection marker by, for example, killing them, sorting them based on fluorescence, or removing them by panning. Examples of negative selection markers that may be used according to the invention include xanthine/guanine phosphoribosyl transferase (gpt), herpes simplex thymidine kinase (HSVtk), and diphtheria toxin A fragment (DTA) (see, e.g., Song, K. Y., et al. (1987) PROC. NAT'L ACAD. SCI. U.S.A. 84, 6820-6824). When included within a vector of the invention, negative selection markers are generally included in addition to a reporter or positive selection marker. Procedures for selecting and detecting markers are widely available and published in the art, including, for example, in Joyner, A. L., GENE TARGETING: A PRACTICAL APPROACH, 2nd ed., (2000), Oxford University Press, New York, N.Y.

Examples of reporters widely used in detecting the presence of a vector include the E. coli β-galactosidase gene (lacZ), which is detected using an enzymatic assay with a substrate such as X-gal, the human placental alkaline phosphatase gene (HPAP), which is detected by an enzymatic assay using a substrate such as BM Purple AP Substrate (Boehringer Mannheim), and green fluorescent protein (GFP), and variants thereof (e.g. EGFP (Clontech Inc.), EYFP, and ECFP. In addition, glucose phosphate isomerase (GPI) may be used as a marker to detect chimeras by GPI cellulose-acetate electrophoresis.

The expression of positive/negative reporters or selectable markers can be detected using a fluorescent activated cell sorter (FACS) for observing emission of light of a specific wave length. For example, a protein that spontaneously emits light and can serve as reporter as well as a positive/negative selectable marker in FACS analysis, is the Green Fluorescent Protein (GFP) isolated from the bioluminescent jellyfish Aequorea Victoria and variants thereof (e.g. EGFP (Clontech Inc.), EYFP, and ECFP. FACS analysis and FACS sorting make it possible to isolate cells that emit light as well as those that do not. As example, the reporter or selectable marker sequence can include the bacterial β-galactosyltransferase which could be used in combination with a vital stain consisting of a fluorescent dye whose emission spectrum could depend on cleavage of a β-glycosidic structure. Subsequent to staining of live cells with the substrate for β-galactosidase, FACS analysis would be employed preferentially to isolate either expressing or non-expressing cells.

Selectable markers include genes that allow for identification, selection and/or sorting of cells based upon cell surface expression of proteins. Preferentially, the proteins would not normally be expressed at high levels and expression would not interfere or adversely affect the biological properties of the cells. Suitable selectable marker sequences include cell-cell adhesion molecules including ICAMs, cadherins or selections that normally are not expressed on the cell of interest, and which do not cross-react with endogenous ligands. Expression of such markers can be detected using specific antibodies, or other forms of natural ligands, in combination with sorting protocols including panning or FACS. In one example, the marker includes a truncated form of a heterologous IL-3 receptor (swine form in mouse cells, human form in swine cells) that is incapable of transducing a signal into the cell. Expression of this receptor is then monitored using the natural ligand (swine or human IL-3) which is preferably conjugated with a fluorescent dye or an enzyme that detectably converts a chromogenic substrate.

A variety of different selection/selectable markers are available in the art to identify vector integration into genomic DNA. Selectable markers that may be used according to the invention, include, for example, dominant and negative selection markers, as well as positive and negative selection markers. Examples of preferred selectable markers include neomycin phosphotransferase (neo), histidinol dehydrogenase (hisD), hygromycin resistance (hygro), thymidine kinase, blasticidin S deaminase (bsr) and puromycin-N-acetyltransferase (puro). Exemplary markers also include chloramphenicol-acetyl transferase (CAT), dihydrofolate reductase (DHFR), and .beta.-galactosyltransferase. For a list of other mammalian selection markers, see Sambrook, J., et al., MOLECULAR CLONING: A LABORATORY MANUAL, 2nd ed. (2001), Cold Spring Harbor Laboratory Press, Cold Spring Harbor, N.Y. Methods of detecting a suitable selectable marker are available in the art and depend, in part, on the origin of the targeted cell.

Other appropriate selectable markers include fusion proteins comprising reporter, and selectable markers, particularly in-frame fusions between lacZ and selectable markers. For example, the marker comprising an in-frame fusion of lacZ and neo (βGEO) permits direct selection of G418 resistant colonies when integration leads to the generation of a functional lacZ/neo fusion protein. Furthermore, selectable markers include cell surface proteins, including cell adhesion molecules, such as integrins. Preferably, such cell surface protein markers are not expressed or expressed at low levels in target cells.

In another preferred embodiment, markers include gene products that confer both positive and negative selection capabilities, including guanidyl phosphorotransferase that enables growth on hypoxanthine but confers sensitivity to growth on 6-thioguanine. Other appropriate selectable markers include fusion proteins comprising reporter or positive selection markers with negative selection markers. An example is the fusion protein containing the Zeomycin resistance gene and the TK gene. Expression of this fusion protein confers the ability to grow in the presence of zeomycin and sensitivity to growth in the presence of gancyclovir. Another example is the marker comprising an in-frame fusion of TK, lacZ and neo (TKβGEO) that permits direct selection of G418 resistant colonies when integration leads to the generation of a functional TK/lacZ/neo fusion protein and for selection for cells lacking a functional TK/lacZ/neo fusion protein by selection of gancyclovir resistant colonies.

Markers may be comprised of multiple translation units comprising reporter, positive selection markers, or negative selection markers. For example, a marker comprising lacZ that is 5′ to an IRES (IRES) that is 5′ to a neo gene permits expression of a functional lacZ and neo gene if preceded by an operably linked promoter or if integrated 3′ to an operably linked promoter, such as a promoter in a genome of a cell.

Preferably, endogenous promoters within genomic sequences drive marker expression. Alternatively, an exogenous promoter capable of driving marker expression may be included within the vector. In some instances, it may be preferable to include an exogenous promoter capable of driving marker selection to ensure that the marker is expressed at levels adequate for detection or selection. For example, when it is known that a genetic locus contains a weak promoter or no promoter at all, an exogenous promoter may be provided to drive marker expression, thereby facilitating identification or selection. However, if the invention is being employed to detect transcriptionally active genes, for example, it may be preferable to not include an exogenous promoter, so that marker expression occurs only when the marker integrates into transcriptionally active DNA.

In another embodiment, the marker sequence will comprise a sequence that can be identified either by intrinsic properties of a marker such as Green Fluorescent Protein (GFP) or through interaction of the marker with probes. Such probes include reagents can be tethered to transcripts derived from marker sequences, such as reagents that share homology with the marker sequence. Such probes also include reagents that can be tethered to polypeptides derived from marker sequence. Probes may elicit changes in the intrinsic properties of a marker, they may themselves have intrinsic properties that make them easily monitored indicators, or they can be tethered to easily measured indicators, such as fluorescent molecules or polypeptides with enzymatic activity. In another preferred embodiment, the marker sequence will encode a polypeptide product that has domains recognized by biologic or chemical agents, including specific antibodies or chemicals that can be monitored.

The marker DNA can also comprise an alternative spliced exon. In this instance, upon integration of the integration vector into the genomic DNA, the corresponding exon of the genomic DNA is replaced by the alternative spliced exon, e.g., in a two step process in which an integration vector comprising recombinase sites inserts into the genomic DNA, and the recombinase sites are used to exchange the alternative spliced exon in a second, conversion vector. Preferably, the integration vector comprises a marker sequence and the alternative spliced exon. In this instance, the recombinant mRNA comprises coding sequence for the marker protein, which is used for selection of recombinant cells.

In another embodiment of the invention, introduced exogenous DNA may contain one or more additional marker sequences and additional promoters suitable for expression of these marker sequences in host cells. In a preferred embodiment, the additional marker sequence is followed by a polyadenylation sequence. One marker sequence can be used for selection of cells integrating DNA into the genome of a host cell independently of other marker sequences of the same vector.

Promoters

A promoter operably linked to a marker sequence can be selected based on the type of host cell. Transcription promoters are widely known and available to those of skill in the art. Suitable promoters include but are not limited to the ubiquitin promoters, the herpes simplex thymidine kinase promoters, human cytomegalovirus (CMV) promoters/enhancers, SV40 promoters, β-actin promoters, immunoglobulin promoters, regulatable promoters such as metallothionein promoters, adenovirus late promoters, and vaccinia virus 7.5K promoters. The promoter sequence also can be selected to provide tissue-specific transcription.

The integration vector may also comprise a bacterial promoter operably linked to a bacterial selection marker, e.g., EM7, for expansion in bacterial cells.

Selection Molecules and Recognition Sites

Recognition sites are sequences within DNA or RNA or polypeptides that are recognized by a selection molecule. One type of recognition site is comprised of RNA or polypeptide sequence derived entirely from genomic sequences of interest. A second type of recognition site is a hybrid recognition site comprised of RNA sequences derived in part from genomic DNA and in part from vector DNA. A hybrid recognition site occurs in recombinant RNA produce by transcription of a recombinant gene resulting from insertion of an integration vector into a genomic region. If the integration vector was a promoter trapping (5′ gene trap) vector, the recombinant RNA comprises a 3′ hybrid recognition site derived from vector DNA sequence, and a 5′ hybrid recognition site derived from genomic DNA sequence. Alternatively, if the integration vector was a PA trapping (3′ gene trap) vector, the recombinant RNA comprises a 3′ hybrid recognition site derived from genomic DNA sequence, and a 5′ hybrid recognition site derived from vector DNA sequence. In either event the 5′ hybrid recognition site is contiguous with the 3′ hybrid recognition site, and together form the complete hybrid recognition site. A hybrid recognition site can also comprise a polypeptide sequence partly derived from genomic DNA and partly from vector DNA. Preferred selection molecules that recognize the RNA sequence include antisense molecules, ribozymes, RNAi molecules, and analytical probes sharing sequence homology with the RNA. Preferred molecules that recognize polypeptide recognition sequences are antibodies. Recognition sites are used to select for or identify cells with integration vectors incorporated into genomic regions of interest.

Antisense selection molecules: A recognition site for an antisense selection molecule is comprised of RNA sequence that is complementary to an antisense molecule. In an embodiment of the invention, a recognition sequence for an antisense molecule is derived from genomic DNA. In a preferred embodiment, the genomic DNA comprises a gene. In another embodiment, the recognition site for an antisense selection molecule is a hybrid recognition site derived partly from vector DNA, and partly from genomic DNA sequence.

Antisense molecules are oligonucleotides that bind in a sequence-specific manner to nucleic acids, such as RNA or DNA. Antisense technology involves expressing or introducing an antisense molecule that is complementary to sequences found in a particular RNA (e.g., the recognition site) into a cell. In one embodiment of the invention, the antisense molecule comprises DNA or derivatives thereof. In another embodiment of the invention, antisense molecules comprise RNA or derivates thereof. In each case, a preferred antisense molecule composition comprises a sequence region that is complementary, and more preferably, completely complementary to the RNA sequence comprising the recognition site. By associating with a mRNA, the antisense molecule inhibits use of the mRNA for production of the polypeptide product of the gene. (See, e.g., U.S. Pat. No. 5,168,053, U.S. Pat. No. 5,190,931, U.S. Pat. No. 5,135,917; U.S. Pat. No. 5,087,617, and Clusel et al. (1993) NUCL. ACIDS RES. 21:3405-3411, which describe dumbbell antisense oligonucleotides, all of which are hereby incorporated by reference in their entirety.) Without wishing to be bound by a particular theory, antisense technology can be used to control gene expression through interference with binding of regulatory molecules (see Gee et al., In Huber and Carr, MOLECULAR AND IMMUNOLOGIC APPROACHES, Futura Publishing Co. (Mt. Kisco, N.Y.; 1994)) and block translation by inhibiting binding of a transcript to ribosomes.

Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate, phosphate esters, and other such linkages (see, e.g., Agrwal et al., TETREHEDRON LETT. 28:3539-3542 (1987); Miller et al., J. AM. CHEM. Soc. 93:6657-6665 (1971); Stec et al., TETREHEDRON LETT. 26:2191-2194 (1985); Moody et al., NUCL. ACIDS RES. 12:4769-4782 (1989); Letsinger et al., TETRAHEDRON 40:137-143 (1984); Eckstein, ANNU. REV. BIOCHEM. 54:367-402 (1985); Eckstein, TRENDS BIOL. SCI. 14:97-100 (1989); Stein In: OLIGODEOXYNUCLEOTIDES. ANTISENSE INHIBITORS OF GENE EXPRESSION, pp. 97-117, Cohen, Ed, Macmillan Press, (London, (1989)); Jager et al., BIOCHEMISTRY 27:7237-7246 (1988)). Methods of designing and producing antisense molecules to disrupt expression through a particular sequence element are well known in the art. For example, antisense molecules have been utilized with polygalactauronase and muscarine type 2 receptor (U.S. Pat. Nos. 5,739,119 and 5,759,829). Antisense molecules have also been described as therapeutics (e.g., U.S. Pat. Nos. 5,747,470; 5,591,317; 5,783,683).

Selection of antisense compositions specific for a given recognition site is based upon analysis of the sequence of the recognition site and determination of secondary structure, melting temperature (Tm), binding energy, and relative stability. Antisense compositions may be selected based upon their relative inability to form dimers, hairpins, or other secondary structures that would reduce or prohibit specific binding to the recognition site within the recombinant mRNA in a cell. The secondary structure analyses and recognition site selection considerations can be performed, for example, using v.4 of the OLIGO primer analysis software and/or the BLASTIN 2.0.5 algorithm software (Altschul et al., Nucleic Acids Res. 1997, 25(17):3389-402). Methods for delivery of antisense molecules are widely known and available to those of skill in the art. For example, the delivery method employing a short peptide vector, termed MPG (27 residues) may be utilized (Morris et al., Nucleic Acids Res. 1997 Jul. 15; 25(14):2730-6). Antisense recognitions sites or portions of antisense recognition sites known to be effective substrates of antisense may be utilized in embodiments of the invention. For example, antisense molecules have been utilized with MDG1, ICAM-1, and human EGF.

Ribozyme selection molecules: A recognition site for a ribozyme selection molecule is comprised of RNA sequence that is complementary to nucleic acids within a ribozyme selection molecule. In an embodiment of the invention, a recognition sequence for a ribozyme selection molecule is wholly derived from genomic DNA sequence. In a preferred embodiment, the genomic DNA comprises a gene. In another embodiment of the invention, the recognition site for an ribozyme selection molecule is a hybrid recognition site derived partly from vector DNA and partly from genomic DNA sequences.

Ribozymes are RNA-protein complexes that cleave nucleic acids in a site-specific fashion, resulting in specific inhibition or interference with cellular gene expression. Ribozymes have specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). At least six basic varieties of naturally-occurring enzymatic RNA are known presently. Each can catalyze the hydrolysis of RNA phosphodiester bonds in trans under physiological conditions. In general, the enzymatic nucleic acid first recognizes and then binds a target RNA through complementary base-pairing. Once bound to the correct site, the ribozyme enzymatically cleaves the targeted RNA. After a ribozyme molecule has bound and cleaved its RNA target, it is released from that RNA so that it can repeatedly bind and cleave new RNA targets. The ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme.

A preferred ribozyme selection molecule comprises a region that is complementary, and more preferably, completely complementary to its recognition site. In other embodiments, ribozymes are used to regulate gene expression. Ribozymes can be targeted to any RNA transcript comprising a recognition site and can catalytically cleave such transcripts (see, e.g., U.S. Pat. No. 5,272,262; No. 5,144,019; and Nos. 5,168,053, 5,180,818, 5,116,742 and 5,093,246 to Cech et al.). Methods of designing and using ribozymes are known in the art, and are described, for example, in the aforementioned patents, as well as U.S. Pat. No. 5,334,711, No. 5,225,337, No. 5,625,047, No. 5,631,359, No. 6,022,962, International Patent Application No. WO 93/23569 and International Patent Application No. WO94/02595 and references cited within. For example, the enzymatic nucleic acid molecule may be formed in a hammerhead, hairpin, a hepatic δ virus, group I intron, or RnaseP RNA (in association with an RNA guide sequence) or Neurospora VS RNA motif. (Rossi et al. Nucleic Acids Res. 1992 Sep. 11; 20(17):4559-65 Hampel et al. (Eur. Pat. Appl. Publ. No. EP 0360257); Hampel and Tritz, Biochemistry 1989 Jun. 13; 28(12):4929-33; Hampel et al., Nucleic Acids Res. 1990 Jan. 25; 18(2):299-304; U.S. Pat. No. 5,631,359; Perrotta and Been, Biochemistry. 1992 Dec. 1; 31(47):11843-52; Guerrier-Takada et al., Cell. 1983 December; 35(3 Pt 2):849-57; Collins (Saville and Collins, Cell. 1990 May 18; 61(4):685-96; Saville and Collins, Proc Natl Acad Sci USA. 1991 Oct. 1; 88(19):8826-30; Collins and Olive, Biochemistry. 1993 Mar. 23; 32(11):2795-9); and U.S. Pat. No. 4,987,071). Recognitions sites known to be effective substrates for specific ribozyme selection molecules may be utilized in embodiments of the invention.

RNAi selection molecules: A recognition site for a RNAi selection molecule is comprised of RNA sequence that is complementary to nucleic acid sequence within an RNAi molecule. In an embodiment of the invention, the recognition site for an RNAi selection molecule is wholly derived from genomic DNA. In a preferred embodiment, the genomic DNA comprises a gene. In another embodiment of the invention, the recognition site for an RNAi selection molecule is a hybrid recognition site derived partly from vector DNA and partly from genomic DNA sequences.

Short-interfering RNA (siRNA) are believed to suppress gene expression through a highly regulated enzyme-mediated process called RNA interference (RNAi) (Sharp, P A 2001. Genes Dev. 15, 485-490; Bernstein, E. et al., 2001. Nature 409, 363-366; Nykanen, A., et. al., 2001, Cell 107, 309-327; Elbeshir, S M et. al., 2001. Genes Dev. 15, 188-200; Bass, B. 2001. NATURE 411:428-429). RNAi molecules comprise any and all reagents capable of inducing an RNAi response in cells. Included are double-stranded polynucleotides such as those comprising a sense strand and an antisense strand. Also included are polynucleotides comprising hairpin loop sequences, such as shRNAi molecules, and expression vectors that express one or more polynucleotides capable of forming a double-stranded polynucleotide alone or in combination with another polynucleotide. Examples of RNAi molecules include double-stranded RNA (dsRNA) molecules, i.e., RNA:RNA hybrids (sense:antisense strands), RNA:DNA hybrids, DNA:RNA hybrids, and DNA:DNA molecules, all of which are included within embodiments of the invention. Accordingly, it is understood that although many embodiments of the invention are described for use with siRNA, any and all RNAi molecules may be used in the embodiments of the invention.

RNAi molecules may be used to disrupt expression of a RNA of interest. Preferably, the RNA is derived from a gene. Although the exact mechanism of RNAi is not essential to embodiments of the invention, homology between the RNAi molecule and an RNA recognition site appears to be required for RNAi-mediated cleavage of RNA within the RNA recognition site. Double strand RNA (dsRNA) introduced into a cell is digested to yield short interfering RNA (siRNA), typically 21-23 nucleotides in length. In mammalian cells, this process is believed to be mediated by the enzyme DICER (Bernstein, E. et al., 2001. Nature 409, 363-366; Ketting, R F et. al., Genes Dev. 15, 2654-2659). RNAi is believed to involve multiple RNA-protein interactions, in which siRNA combines with an RNA-inducing silencing complex (RISC) that is subsequently activated, recognizes the target region of an RNA through interaction with the RNAi recognition sequence, and cleaves the target RNA within the RNAi recognition site. Introduction of RNAi molecules into a cell is accomplished by using a variety of methods and procedures known in the art. For example, double strand RNA methods and reagents are described in PCT applications WO 99/32619, WO 01/68836, WO 01/29058, WO 02/44321, WO 01/92513, WO 01/96584, and WO 01/75164, which are hereby incorporated by reference. RNAi mediated suppression of mRNA expression was correlated with loss of polypeptide (Caplen, N. et al., PROC. NATL. ACAD. SCI. USA 98:9746-9747 (2001)).

Short hairpin RNAs may also be used as selection molecules according to the invention. Short hairpin RNA (shRNA) is a form of hairpin RNA capable of sequence-specifically reducing expression of a target gene. Short hairpin RNAs may offer an advantage over siRNAs in suppressing gene expression, as they are generally more stable and less susceptible to degradation in the cellular environment. Such shRNA-mediated gene silencing works in a variety of normal and cancer cell lines, and in mammalian cells, including mouse and human cells. Paddison, P. et al., 2002. GENES DEV. 16(8):948-58; Berns K. et al., 2004. Nature 428, 431-437; Paddison, P J etl al., 2004. Nature 428, 427-431. ShRNAs contain a stem loop structure. In certain embodiments, shRNA may contain variable stem lengths, typically from 19 to 29 nucleotides in length, from 19 to 21 nucleotides in length, or from 27 to 29 nucleotides in length. In certain embodiments, loop size is between 4 to 23 nucleotides in length, although the loop size may be larger than 23 nucleotides without significantly affecting silencing activity. ShRNA molecules may contain mismatches, for example G-U mismatches between the two strands of the shRNA stem without decreasing potency. In certain embodiments, shRNAs include one or several G-U pairings in the hairpin stem to stabilize hairpins during propagation in bacteria. In a preferred embodiment of the invention, complementation and preferably complete complementation between the portion of the stem that binds to the recognition site is desired, since even a single base pair mismatch in this region may abolish silencing. 5′ and 3′ overhangs are not required, since they do not appear to be critical for shRNA function, although they may be present (Paddison et al. (2002) GENES & DEV. 16(8):948-58).

A number of structural characteristics of effective siRNA selection molecules have been identified. Elshabir, S. M. et al. (2001) NATURE 411:494-498 and Elshabir, S. M. et al. (2001), EMBO 20:6877-6888. Accordingly, one of skill in the art would understand that a wide variety of different siRNA selection molecules may be used to target a specific gene. In certain embodiments, siRNA selection molecules are 16-30 or 18-25 nucleotides in length. In a preferred embodiment, siRNA is 21 nucleotides in length. In certain embodiments, the siRNAs have 0-7 nucleotide 3′ overhangs or 0-4 nucleotide 5′ overhangs. In a preferred embodiment, an siRNA molecule has a two nucleotide 3′ overhang. In another preferred embodiment, an siRNA is 21 nucleotides in length with two nucleotide 3′ overhangs (i.e. they contain a 17 nucleotide complementary region between the sense and antisense strands. In certain embodiments, the overhangs are UU or dTdT 3′ overhangs. Generally, siRNA molecules are complementary and preferably completely complementary to a recognition site in a recombinant RNA molecule, since even single base pair mismatches have been shown to reduce silencing. In other embodiments, siRNAs may have a modified backbone composition, such as, for example, 2′-deoxy- or 2′-O-methyl modifications. However, in preferred embodiments, the entire strand of the siRNA is not made with either 2′ deoxy or 2′-O-modified bases. Potential RNAi selection molecule sequences may be compared to an appropriate genome database, such as BLAST, available on the NCBI server at www.ncbi.nlm, to optimize for binding to recognition sequence within the genomic sequence.

Algorithms for selection of recognition sites and RNAi selection molecules are implicated in embodiments of the invention. For example, using a nucleotide numbering system based on use of a 21 base pair siRNA molecule with a 2 base pair, 3′ overhang on each end of the molecule, at least eight criteria for overall effectiveness have been identified (Reynolds, A. 2004. Nature Biotechnology 22, 326-330). These criteria include:
I. 30% to 50% G/C content
II. At least 3 ‘A/U’ bases at positions 15-19 (sense strand)
III. Absence of internal repeats (Tm of potential internal hairpin is xxx
IV. An “A” base at position 19 (sense strand)
V. An ‘A’ base at position 3 (sense strand)
VI. A ‘U’ base at position 10 (sense strand)
VII. A base other than ‘G’ of ‘C’ at position 19 (sense strand)
VIII. A base other than ‘G’ at position 13 (sense strand)
   Using these criteria, an algorithm (identified here as the “Reynolds” algorithm) was established, as follows: Meeting criteria I, III, IV, V and VI was assessed 1 point each. Failure to satisfy criteria VII and VIII was assessed −1 point each. For criterion II, 1 point each was assessed for each A or U at positions 15-19. The scores for siRNA molecules can therefore range between −2 to 10. In a systematic evaluation of 180 siRNA molecules directed at 2 genes, algorithm scores greater than 6 (e.g. 15.5% of the panel) were highly predictive of siRNA activity. For example, 100% exhibited silencing >50%, 92.5% greater than 80%, and 46.4% greater than 95%. Another algorithm that may be used in embodiments of the invention are described by Kumiko, U T et. al., 2004. Nucleic Acids Res. 32, 936-948.

In the embodiment of the invention involving a recognition site for an RNAi selection molecule that is wholly derived from sequences that reside in genomic DNA. In a preferred embodiment, RNAi molecules are comprised of sequences homologous to those residing in genomic DNA, corresponding to a recognition site of a recombinant RNA molecule. In another embodiment of the invention, an algorithm, such as the Reynolds algorithm, is used to identify efficacious sequences in the genome for designing RNAi selection molecules. In embodiments of the invention, these RNAi selection molecules are used to suppress expression of a recombinant RNA molecules comprised of sequences derived from genomic and vector DNA. For example, a score of at least 5, preferably 6, more preferably 7, most preferably 8-10 criteria set forth in the above algorithm will be used to design RNAi selection molecules.

The other embodiment of the invention involves a hybrid recognition site derived partly from genomic DNA and partly from vector DNA sequences. For example, an integration vector comprising a SA (SA) may be introduced into a mammalian cell, such as a human cell. When integrated into an intron of a gene, splicing occurs between the SA site in the vector and a SD site in genomic DNA. Through splicing, a recombinant mRNA molecule is formed, comprising RNA derived from genomic (exon)-DNA residing 5′ to the vector insertion site into the genome and comprising RNA derived from vector sequences residing 3′ to the SA. Accordingly, this recombinant mRNA comprises a hybrid recognition site at the junction of the genome-derived and vector-derived mRNA in which sequences of the hybrid recognition site residing 5′ to the junction are derived from genomic DNA while those residing 3′ to the junction are derived from vector DNA. Similarly, an integration vector comprising a SD may be introduced into a mammalian cell, such as a human cell. When integrated into an intron of a gene, splicing occurs between the SD site in the vector and a SA site in genomic DNA. Through splicing, a recombinant mRNA molecule is formed, comprising RNA derived from vector sequences residing 5′ to the SD site and genomic (exon)-DNA residing 3′ to the vector insertion site. Accordingly, this recombinant mRNA comprises a hybrid recognition site at the junction of the genome-derived and vector-derived mRNA in which sequences of the recognition site residing 5′ to the junction are derived from vector DNA while those residing 3′ to the junction are derived from genomic DNA.

In an embodiment of the invention, criteria from algorithms will be utilized to design sequences of a hybrid recognition site for an RNAi selection molecule sufficient for suppression of the recombinant mRNA by 25%, more preferably 50%, more preferably 60%, more preferably 70%, more preferably 80%, more preferably 90%, more preferably 95%, and most preferably more than 99%. For example, the Reynolds algorithm is used to design efficacious sequences for the hybrid recognition site. For example, a score of at least 5, preferably 6, more preferably 7, most preferably 8-10 criteria set forth in the above algorithm will be used to design RNAi selection molecules. Criteria are met by 1) determining the number of hybrid recognition site nucleotides that will be derived from vector versus the number derived from genomic DNA, 2) identifying a preferred genetic locus of interest comprising a SA or SD site, and 3) determining the nucleotide sequence vector determinants of the hybrid recognition site. For example, although the sequence of the vector DNA can be entirely controlled, sequences residing within exons are typically fixed in host cells. Therefore, a variable for determining the number of nucleotides of the hybrid recognition site that are derived from the vector is the particular sequence of genomic DNA that will be included in the hybrid recognition site. In particular, altering the number of nucleotides derived from vector may impact total GC content of the hybrid recognition site (Reynolds criteria # 1), the absence of internal repeats (Reynolds criteria #3), and the presence or absence of an ‘A’ at nucleotide 3 of the sense strand (Reynolds criteria # 5; in the case in which a SA site is utilized in the vector). These same criteria for a hybrid recognition site can be impacted by choice of insertion site, such as a different intron of a gene. Moreover, when a SA is utilized, at least 5 out of the eight Reynolds criteria, ensuring a score of at least 7 out of 10 possible points, are entirely controlled by sequences within the vector; Reynolds criteria 2, 4, 6, 7, and 8. Rather than using algorithms and genome sequence data to derive the sequence of a hybrid recognition site, an embodiment of the invention is to utilize portions of specific recognition sites for known RNAi molecules within integration vectors of the invention for the same purpose. The hybrid recognition sites in these cases are partly derived from vector sequences derived from recognition sites for known RNAi molecules and partly from genomic DNA. In embodiments of the invention, these RNAi selection molecules are used to suppress expression of an RNA molecule comprised of sequences derived from genomic and vector DNA.

Analytical Probes: Single stranded or double stranded RNA or DNA molecules may be used as selection molecules in various embodiments of the invention. In one embodiment of the invention, a recombinant RNA of the invention is derived wholly from DNA sequence of a gene, or derived partly from gene sequence and partly from vector DNA sequence. The analytical probe is complementary (or homologous) to a recognition site in the recombinant mRNA. The recognition site can be a hybrid recognition site derived from genomic and vector DNA. The analytical probe is used to detect cells with vector integrated into a desired genetic locus and that produce recombinant RNA transcripts consisting of gene-derived sequence and vector-derived sequence. The nucleic acid sequence present selection molecule is substantially complementary (or homologous) when it is more than 60%, preferably more than 70%, even more preferably more than 80%, highly preferably more than 90%, such as 95%, and most preferably more than 99% complementary (or homologous) to the sequence of the recognition site. Methods for designing and synthesizing single stranded RNA or DNA probes are known in the art, as are methods for hybridizing to target RNA samples.

Antibody selection molecules: Either monoclonal or polyclonal antibodies may be used as selection molecules in various embodiments of the invention. In one embodiment of the invention, a recombinant RNA of the invention is translated into a polypeptide comprised of an amino acid sequence derived wholly from genomic DNA sequence, or derived partly from genomic DNA and partly from vector DNA sequence. An antibody specific to a hybrid recognition site in this polypeptide, e.g., an antibody whose antigenic sequence is comprised of amino acids derived from genomic and vector DNA, is used to detect for cells with vector integrated into a desired genetic locus. Methods for designing and synthesizing polypeptides for antibody production are known in the art, as are methods for production of polyclonal and monoclonal antibodies.

Homologous Domains

In another embodiment of the invention, the integration vector preferably comprises homologous domains that flank the marker sequence. Homologous domains are comprised of polynucleotides with sequences that are substantially homologous to those residing within target loci in a host cell genome. These domains function during homologous recombination whereby the homology domains target the integration vector to recombine into the target genomic region. Homologous recombination is the process of DNA recombination based on sequence homology between nucleic acid sequences in the integration vector with nucleic acid sequences in the target genomic region. Any nucleic acid sequence sufficient to promote homologous recombination into the target genomic region may be used in embodiments of the invention. The nucleic acid sequences present in the integration vector are substantially homologous when they are more than 60%, preferably more than 70%, even more preferably more than 80%, highly preferably more than 90%, such as 95%, and most preferably more than 99% homologous to the nucleic acid sequence of a target genomic region.

Methods for preparing homology domains are known in the art and include cloning of sequences derived from genomic DNA, PCR of sequences derived from genomic DNA, or synthesis of sequences homologous to genomic DNA. Methods for combining homology domains to integration vector components include restriction digestion and ligation.

Splice Acceptor Site (SA)

SAs are nucleotide sequences recognized in pre-processed RNA by the spliceosome. SAs are typically located at the 3′ ends of introns, but cryptic SAs may also be found throughout the genomes of cells. SA sequences facilitate excision and splicing reactions. SA and SD are used in RNA splicing reactions to adjoin flanking RNA that resides 5′ to a SD site and flanking RNA that resides 3′ to a SA site. RNA splicing reaction typically forms the junction between exons. SA sequences are widely known and available to those of skill in the art. SA sequences can be derived from exons of cellular genes, including exons that are expressed constitutively as well as ubiquitously including β-actin, PGK-1, and HPRT. Preferably, SA sequences are not derived from a region of DNA that includes the 5′-splice junction of exons which are subject to alternative splicing, either tissue specifically, cell-type specifically or stage specifically. SA sequence may include around 100 nucleotides of the 5′ splice junction of the adenovirus SV40. In a preferred embodiment, SA sites within vectors of the invention are comprised of a functional SA consensus sequences. Preferably, the SA comprises a pyrimidine-rich region, preceding the dinucleotide AG. For instance, a suitable SA may be NTN(TC)(TC)(TC)TTT(TC)(TC)(TC)(TC)(TC)(TC)NCAGG. Optimizing the SA sequence used can further enhance, or regulate, the efficiency of the 5′ gene trap cassette.

SAs in vectors of the invention are preferably used to splice vector sequences residing 3′ to the SA with genomic DNA sequences, such as exon sequences, that reside 5′ to a SA that resides up-stream (5′) of the SA of the vector.

Splice Donor Site (SD)

SDs are nucleotide sequences recognized in pre-processed RNA by the spliceosome. SDs are typically located at the 5′ ends of introns, but cryptic SDs may also be found throughout the genomes of cells. SD sequences facilitate excision and splicing reactions. SA sites and SDs are used in RNA splicing reactions to adjoin flanking RNA that resides 5′ to a SD and flanking RNA that resides 3′ to a SA site. RNA splicing reaction typically forms the junction between exons. SD sequences are widely known and available to those of skill in the art. SD sequences can be derived from exons of cellular genes, including exons that are expressed constitutively as well as ubiquitously including β-actin, PGK-1, and HPRT. Preferably, SD sequences are not derived from a region of DNA that includes the 5′-splice junction of exons which are subject to alternative splicing, either tissue specifically, cell-type specifically or stage specifically. In a preferred embodiment, SD sites within vectors of the invention are comprised of a functional SD consensus sequences. An example of a suitable SD is NAGGT(AG)AGT. SD sites in vectors of the invention are preferably used to splice vector sequences residing 5′ to the SD site with genomic DNA sequences, such as exon sequences, that reside 3′ to a SA that resides down-stream (3′) of the SD site of the vector.

Internal Ribosome Entry Site (IRES)

To increase the probability for translation of the marker sequence to occur in the proper frame, an IRES is included to initiate translation of an internal open reading frame (ORF) within a recombinant mRNA, including polycistronic mRNA. Intron/exon junctions do not correspond or follow a particular rule related to the conservation of the ORF, i.e., junctions breakpoints may be at any position within a codon. Provided that any one position of a codon can include the intron/exon junction, marker sequences whose ORF starts at that junction would be translated in the proper frame at an approximate probability of ⅓ only.

An IRES directs attachment of a downstream coding region or of an ORF with a cytoplasmic polysomal ribosome by initiating translation in the absence of any internal promoters. In a preferred vector, the IRES is included to initiate translation of marker coding sequences. Examples of suitable IRES that can be used to practice the invention include the mammalian IRES of the immunoglobulin heavy-chain-binding protein (BiP), picornaviruses IRES, e.g., encephalomyocarditis virus (preferably nucleotide numbers 163-746), poliovirus (preferably nucleotide numbers 28-640) and foot and mouth disease virus (preferably nucleotide numbers 369-804). Examples of suitable IRES sequences are disclosed in European patent application 585983 and PCT applications WO/9611211, WO/9601324, and WO/9424301. Particularly preferred is the IRES from GTX homeoprotein (see Chappell et al. (2004) Proc Natl Acad Sci USA., 101:9590-94; and Owens et al. (2001) Proc Natl Acad Sci USA., 98:1471-76; Hu, M. C.-Y. et al, Proc. Natl. Acad. Sci USA 96:1339-1344 (1999); , all of which are incorporated by reference).

Polyadenylation Site (PolyA)

In certain embodiments, vectors of the invention include a transcription termination sequence that is 3′ of the marker. A transcription termination sequence is any polynucleotide sequence whose presence within a gene disrupts or terminates transcription of the gene. Transcription termination sequences include, for example, polyadenylation (PA) sequences and the trans-acting responsive (TAR) element present just 3′ to the start site of transcription in the human immunodeficiency virus LTR. Binding of the viral protein tat and cellular proteins to the TAR allows full-length transcription of the retroviral genome. The presence of the TAR within the HIV LTR promoter essentially renders the promoter inactive in the absence of the tat gene product, which is expressed upon viral infection. A variety of elements and molecules regulating transcription termination are well known in the art and have been described, for example, in Zhao, J. et al., FORMATION OF MRNA 3′ ENDS IN EUKARYOTES: MECHANISM, REGULATION, AND INTERRELATIONSHIPS WITH OTHER STEPS IN MRNA SYNTHESIS, (1999), Microbiology and Molecular Biology Reviews, 63, 405-445. Any regulatable transcription termination sequence may be used according to the invention, and alternative transcription termination sequences may be used in place of PA sites. In preferred embodiments, the introduction of the transcription termination sequence into the disrupted endogenous gene will result in the expression of a RNA transcript with a 3′ end determined by the transcription termination sequence. Polyadenylation sites include, for example, the SV40 PA site, the phosphoglycerate kinase PA site, and the bovine growth hormone PA site, described in the Invitrogen 1996 catalog and Joyner, supra. The addition of a PA site may be employed to enhance expression of the marker sequences in recombinant cells (Thomas, K., et al., Cell 44:419-428 (1986)). Transcription termination sequences are widely known and available to those of skill in the art.

Recombinase

Suitable recombinase sites include FRT sites and loxP sites, which are recognized by the flp and cre recombinases, respectively (See U.S. Pat. No. 6,080,576, No. 5,434,066, and No. 4,959,317). Other elements, such as transposable elements and recombinase recognition sequences, also may be added to the trap construct and used in a similar fashion to the FRT-flp and loxP-cre system described herein. For example, suitable recombinase sites also include lambda recombinase sites as described in U.S. Pat. Nos. 5,888,732, 6,143,557, 6,171,861, 6,270,969, and 6,277,608. The Cre-loxP and Flp-FRT recombinase systems are comprised of two basic elements: the recombinase enzyme and a small sequence of DNA that is specifically recognized by the particular recombinase. Both systems are capable of mediating the deletion, insertion, inversion, or translocation of associated DNA, depending on the orientation and location of the target sites. Recombinase systems are disclosed in U.S. Pat. No. 6,080,576, No. 5,434,066, and No. 4,959,317, and methods of using recombinase systems for gene disruption or replacement are provided in Joyner, A. L., Stricklett, P. K. and Torres, R. M. and Kuhn, R. In LABORATORY PROTOCOLS FOR CONDITIONAL GENE TARGETING (1997), Oxford University Press, New York.

Representative minimal target sites for Cre and Flp are each 34 base pairs in length and are known in the art. The orientation of two target sites relative to each other on a segment of DNA directs the type of modification catalyzed by the recombinase: directly orientated sites lead to excision of intervening DNA, while inverted sites cause inversion of intervening DNA. In certain embodiments, mutated recombinase sites may be used to make recombination events irreversible. For example, each recombinase target site may contain a different mutation that does not significantly inhibit recombination efficiency when alone, but nearly inactivates a recombinase site when both mutations are present. After recombination, the regenerated recombinase site will contain both mutations, and subsequent recombination will be significantly inhibited.

Recombinases useful in the present invention include, but are not limited to, Cre and Flp, and functional variants thereof, including, for example, FlpL, which contains an F70L mutation, and Flpe, which contains P2S, L33S, Y108N, and S294P mutations. Cre or Flpe is preferably used in ES cells, since they have been shown to excise a chromosomal substrate in ES cells more efficiently than FlpL or Flp (Jung, S., Rajewsky, K, and Radbruch, A., (1993), SCIENCE, 259, 984). Optionally, the marker sequence in the integration vector can be flanked by suitable recombinase sites. These latter sites are useful for integrating additional DNA sequences or to replace integration vector sequences, either in cell free reactions or in living cells, such as bacteria or eukaryotic cells (e.g., lox P and frt).

In one embodiment, a first vector comprising a recombinase site flanking a marker sequence (e.g., an marker sequence integrated into genomic DNA) is used in conjunction with a second accessory vector that comprises a different detectable marker, selectable marker, or an enzymatic marker, and that is preferably not flanked by the same recombinase sites the first vector.

In the event that both of the 5′ gene trap cassettes are not expressed at acceptable levels (via alternative splicing), the second 5′ gene trap cassette (that encodes a detectable marker) can be “activated” by using a suitable recombinase activity (i.e., cre, flp, etc.) in vitro or in vivo to remove the first (recombinase site flanked) 5′ gene trap cassette.

FRT comprise different 8 base pair core sequences. Alternate pair sites, e.g., here designated A and B, do not recombine with each other as well as they do to other FRT sites with similar core sequences. Preferably, these are used in combination to minimize intramolecular recombination (e.g., excision) when translocating DNA.

Introduction of Integration Vector into Cells

The integration vectors can be incorporated into a bacterial plasmid, or a viral vector, such as a retrovirus, adenovirus or adeno-associated virus vector, for efficient delivery to eukaryotic cells. The recombinant vector can transduce dividing cells, and upon infection, can integrate its genome at random sites in chromosomal DNA of host cells. Methods for introducing DNA into host cells are known in the art. Essentially, any method for introducing DNA into a cell in which the introduced DNA is incorporated into the genome can be utilized. Preferably, integration is stable for more than 24 hours after introduction. More preferably, integration is stable for more than 4 days. Even more preferably, integration is stable for more than 2 weeks. The vectors of the invention may comprise other components that enable amplification of vector sequences. The vectors of the invention may also comprise components that facilitate methods for introduction of vector sequences into host cells, such as eukaryotic cells, plant cells, mammalian cells, mouse cells, and human cells. A retroviral vector will have LTRs derived from one or several types of retroviruses, and the LTRs may be genetically modified to achieve desired properties in the cell type of interest such as. For example, the LTRs may be self inactivating so that promoter activity is suppressed or inactive in the host cell (Yu, S. F. et. al., 1986. PNAS 83(10):3194-8). Genetic modifications may also achieve desired properties in a stem cell or an embryonic stem cell derived from mouse, pig or human, or including a hematopoietic stem cell derived from various mammalian origins.

The integration vector can also include regulatory elements suitable for propagation, amplification, and selection in a suitable cell, such as E. coli. Regulatory elements for propagation, amplification and selection include an origin of replication (ori) and an antibiotic resistance marker for selection (Amp^{R}). In an embodiment of the invention, the resistance marker for selection is the Neomycin phosphorotransferase gene (neo) that confers bacterial resistance to Kanamycin. A bacterial promoter may also be included, to express selection markers.

Alternatively, delivery of the integration vector into a host cell can be performed using electroporation. Electroporation is a feasible approach for delivery of the integration vector to certain types of cells including mouse cells, human cells, human cell lines, embryonic stem cells or hematopoietic stem cells. Generally, the efficiency of generating stable transformants of eukaryotic cells is somewhat lower than with retroviral vectors, but is preferable in cases where the cells are refractory to viral infection or integration of the provirus into the host chromosome.

Delivery of the integration vector into host cells can also be achieved by liposome-mediated transfection, calcium phosphate precipitation as well as DEAE-dextran or other techniques well known to those in the field. See Sambrook and Maniatis (1989). See CURRENT PROTOCOLS IN MOLECULAR BIOLOGY, (2001), Ausubel et al. (eds.), John Wiley & Sons, New York and Sambrook, et al., MOLECULAR CLONING: A LABORATORY MANUAL, (2001), Cold Spring Harbor Laboratory Press, Cold Spring Harbor, N.Y., and U.S. Pat. No. 5,789,215, for example. Lipofection can also be used so that the gene trap vector will become translocated across the plasma and nuclear membrane for stable integration into random sites of the chromosomes from cell types that are permissive for lipofection, including mouse embryonic stem cells. In a preferred embodiment of the invention, integration vectors may be linearized, such as with restriction endonucleases, prior to introduction into host cells.

The integration vector can be introduced to ecotropic producer cell lines to yield virus that infects mouse cells only. Furthermore, the viral trap vector can be packaged in amphotropic producer cell lines including AM 12 or PA317 (Miller and Rosman, 1989) to yield virus that can infect human or porcine cells, for example.

Methods for Identifying Cells Containing Gene-Specific, Recombinant RNA

An embodiment of the invention is to provide methods for identifying cells in which an integration vector of the invention has integrated into a genetic locus or gene of interest. In an embodiment of the invention, an integration vector is introduced into a population of host cells in which the vector integrates randomly into the genome. As result of integration, the host cells express a recombinant RNA comprised of sequences derived from genomic DNA and sequences derived from vector DNA. In an embodiment of the invention, cells integrating into desired genetic loci or genes are identified by introducing into the cells a selection molecule. On the one hand, a selection molecule wholly recognizes genome-derived sequences and thereby suppresses expression of the recombinant RNA. Identification of cells with integrated vector is accordingly performed by selection methods or screening methods to identify cells with suppressed expression of marker sequences comprising the recombinant RNA or derived from the recombinant RNA. On the other hand, a selection molecule is a reagent that specifically recognizes a polynucleotide or polypeptide that is specific to the recombinant RNA. For example, when a SA or SD is included in the integration vector and when the integration vector integrates into an intron of a gene, the splicing machinery of the cell may adjoin RNA derived from genomic sequences with RNA derived from vector sequences. Sequences at the junction between genome derived and vector derived RNA is not found in the wild type cell genome, nor the vector DNA, and is specific to the site of integration. Sequences at the junction accordingly represent a gene-specific marker which can be recognized by selection molecules of the invention. Alternatively, sequences at the junction may be translated into polypeptides comprised of amino acid sequences derived from genomic DNA and amino acid sequences derived from vector DNA. The unique polypeptide sequence at the junction of these regions are are also specific to the particular recombinant cell and genetic locus and may be identified by use of a selection molecule, such as an antibody. Additional methods for monitoring expression or the presence or absence of gene-specific, recombinant RNA includes use of PCR, northern analysis, and Southern analysis, methods of which are commonly used and known in the art. Additionally, methods using complementary polynucleotides, such as in situ hybridization may be utilized.

In a preferred embodiment, the selection molecule suppresses expression of a marker. In a preferred embodiment, the marker is derived from expression of vector sequences. In another preferred embodiment, the marker is derived from expression of sequences derived from the genome in combination with sequences derived from the vector. In another preferred embodiment, the selection molecule is an antisense molecule. In another preferred embodiment, the selection molecule is a ribozyme. In a highly preferred embodiment, the selection molecule is an RNAi molecule. In a preferred embodiment, the selection molecule is a molecule comprised of nucleic acid sequences that are complementary to the recombinant RNA. In a preferred embodiment, the selection molecule is a molecule comprised of nucleic acid sequences that are complementary to RNA sequences derived from genomic DNA and RNA sequences derived from vector DNA. In more preferred embodiment, the selection molecule is a molecule comprised of nucleic acid sequences that are complementary to the junction of genome-derived and vector-derived RNA sequences of recombinant RNA. In another preferred embodiment, the selection molecule is a reagent that recognizes a polypeptide derived from expression of the recombinant RNA. In another embodiment, the selection molecule is a reagent that recognizes expression of amino acids derived from the recombinant RNA that further comprise the junction of amino acids derived from genomic sequences and amino acids derived from vector sequences. In another embodiment, the selection molecule is a reagent the recognizes expression of amino acids at the junction of genome-derived and vector-derived sequences. In another embodiment, the selection molecule is an antibody. In another embodiment, the selection molecule is a chemical reagent.

In a preferred embodiment of the invention, expression of the recombinant RNA and marker are facilitated by an endogenous promoter. In a preferred embodiment of the invention, cells integrating an integration vector are first screened for expression of a marker sequence that is dependent on expression from an endogenous promoter. In another preferred embodiment of the invention, expression of the recombinant RNA and marker are mediated by a promoter within the introduced vector. In a preferred embodiment of the invention, cells integrating an integration vector are first screened for expression of a marker sequence that is dependent on expression from a vector promoter.

Following exposure to environmental perturbations known to inhibit expression of a gene of interest, cells are screened to identify those with reduced marker expression. The recovered cells have enhanced probability to contain exogenous DNA integration into the desired genes and loci. In a preferred embodiment, the known inhibitors of gene expression are biological agents, such as ligands for receptors or chemical agents known to inhibit expression of the desired gene. The known inhibitor may be a natural or artificial transcription factor that has some level of specificity for the desired gene. In a preferred embodiment, the inhibitor is an antisense molecule, a ribozyme, or an agent that induces an RNA interference response that has some level of specificity for the desired gene.

In another embodiment, cells integrating a integration vector comprising a vector promoter are first selected for cells expressing a marker sequence. Following exposure to environmental perturbations known to inhibit expression of a desired gene, cells are screened to identify those with reduced marker expression. The recovered cells have enhanced probability to contain exogenous DNA integration into the desired genes and loci. In a preferred embodiment, the known inhibitor is an antisense molecule, a ribozyme, or an agent that induces an RNAi response that has some level of specificity for the desired gene.

Targets

Integration vectors and selection molecules of the invention may be used to target genetic loci, including transcribed and nontranscribed genomic DNA. Included are endogenous genes, transgenes, and genes of a pathogen in the cell. The present invention is not limited to any particular type of genetic locus or nucleotide sequence. However, exemplary classes of target genes include developmental genes (e.g., adhesion molecules, cyclin kinase inhibitors, Writ family members, Pax family members, Mad family members, Winged helix family members, HOX family members, nuclear hormone receptor family members, cytokines/lymphokines and their receptors, growth/differentiation factors and their receptors, neurotransmitters and their receptors), oncogenes (e.g., ABL1, BCL1, BCL2, BCL6, CBFA2, CBL, CSFIR, ERBA, ERBB, ERBB2, ETSI, ETS1, ETV6, FGR, FOS, FYN, HCR, HRAS, JUN, KRAS, LCK, LYN, MDM2, MLL, MYB, MYC, MYCLI, MYCN, NRAS, PIM1, PML, RET, SRC, TALI, TCL3 and YES); tumor suppressor genes (e.g., APC, BRCA1, BRCA2, MADH4, MCC, NF1, NF2, RB1, TP53 and WT1); enzymes (e.g., ACC synthases and oxidases, ACP desaturases and hydroxylases, ADP-glucose pyrophorylases, ATPases, alcohol dehydrogensases, amylases, amyloglucosidases, catalases, cellulases, chlcone synthases, GTPases, helicases, hemicellulases, integrases, inulinases, invertases, isomerases, kinases, lactases, lipases, lipoxygenases, lysozymes, nopaline synthases, octopine synthases, pecinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases); apoptosis genes (e.g., bcl genes, Ced-3, human ICE (interleukin-1-(3 converting enzyme) (caspase-1), ICH-1 (caspase-2), CPP32 (caspase-3), ICErelll (caspase-4), ICErellll (caspase-5), Mch2 (caspase-6), ICE-LAP3 (caspase-7), Mch5 (caspase8) ICE-LAP6 (caspase-9), Mch4 (caspase-10), caspases 11-14, and others).

### EXAMPLES

### Example 1

### Reagents Comprising a 5′ Gene Trap Vector and Methods for RNAi Regulation of Marker Expression

A) Synthesis of Core Gene Trap Vectors:

The homologous integration vectors are typically comprised of a gene trap “core” flanked on each end by sequences homologous to the targeted integration site. Identification and recovery of recombinant cells with marker DNA integrated at the target locus resides primarily with development of reagents and methods that utilize elements that reside within the gene trap core of the vector. The functionality of all gene trap elements of the proprietary vector is tested.

A gene trap core is first constructed or synthesized. Synthesis of the core vector is preferred, since vector components are complex, restriction sites are introduced and removed for ease of certain gene identification applications and for future vector developments. Synthesis enables reduction of methylation sites (CG-s) in the vector that upon prolonged cell culture can lead to suppression of reporter activity. Synthesis allows alteration of degenerate codon usage to maximize utilization of human tRNAs thereby optimizing expression and hence sensitivity of the reporter.

From 5′ to 3′, elements of a preferred gene trap core includes a flip recombinase target sequence (FRT), a SA, a 3′ hybrid recognition site, translation stop codons in all 3 reading frames (STOP), a bacterial EM-7 promoter (EM-7), an IRES (GTX), TKβGEO, a PA, a ORI, and a second FRT sequence.

B) Introduction of Core Gene Trap Vectors into Human Cells, and Selection for Cells Integrating Vector into Transcribed DNA

Since a mammalian promoter is not included in the vector, upon integration into non-transcriptionally active DNA, expression of the marker TKβGEO gene does not occur. Because cells do not express Neo as part of the TKβGEO fusion, they are sensitive to the antibiotic G418. In contrast, if the vector integrates into an intron of a transcribed gene, the “trapped” promoter of the endogenous gene drives expression of a recombinant primary transcript product comprised of endogenous (trapped) DNA 5′ to genomic insertion site and vector sequences residing 5′ to the poly-A site. Included in this recombinant gene transcript are the FRT, SA, a hybrid recognition site, STOP, GTX, EM-7, and TKβGEO components.

In mammalian cells, the primary gene transcript is processed by the splicing machinery of the cell to generate a recombinant mRNA comprised of endogenous exon sequences located 5′ to the SA and vector sequences residing 3′ to the SA and 5′ to the poly-A site. Included in the mRNA are the hybrid recognition site, STOP, EM-7, GTX, and TKβGEO components. The endogenous promoter of the trapped gene drives expression of mRNA in which translation of exon-derived mRNA terminates at the vector derived translation termination sites (STOP) and in which ribosomes initiate translation from the GTX IRES to express the TKβGEO fusion gene. Such gene trapped cells are resistant to G418. Selection of cells in which plasmid sequence has integrated into transcriptionally active DNA is accomplished by selection on G418. Recovery of G418 resistant cells validates the functionality of the SA, the GTX IRES, and the Neo portion of the TKβGEO fusion gene.

C) Evaluation of β-Galactosidase Activity to Validate β-Gal Functionality

Gene trapping places expression of the marker TKβGEO gene under regulation of the endogenous promoter. Integration of the marker sequence produces reporter cells for the gene into which the vector has integrated, by virtue of having β-galactoside activity under regulation of the endogenous promoter. The functionality of the β-galactosidase functionality is shown by cloning G418 resistant cells, which express β-galactosidase activity.

D) Evaluation of Sensitivity to Gancyclovir to Validate TK Functionality

Thymidine kinase (TK) is expressed in the gene-trapped cells as part of the TKβGEO fusion gene. TK is a negative selectable marker, in that cells expressing TK are sensitive to gancyclovir. The functionality of TK activity is shown by the sensitivity of gene-trapped cells to gancyclovir as compared to wild type cells.

E) Cloning of Cells and Identification of Vector Integration Sites into Genomic DNA by Gene-Tagging (Plasmid Rescue), Sequencing, and Alignment with a Human Genome Database

Vector insertion sites into genomic DNA are determined for a number of G418 resistant clones. A gene-tagging (plasmid rescue) methodology is utilized (Hicks, G G et. al. 1997. Nature Genetics 16, 338-344). For example, restriction endonucleases cleave genomic DNA isolated from clones. The restriction endonuclease sites are not found within vector DNA (e.g., EcoR1 which cuts genomic DNA every 2-3 Kb on average). Restriction cut genomic DNA is then self-ligated using DNA ligase to form closed circular DNA molecules. Included in the preparations are recombinant DNA molecules comprised of DNA derived from vector linked with immediately flanking genomic DNA. The ligated DNA preparations are then introduced into bacteria where the EM-7 promoter drives expression of TKβGEO and the ORI amplifies the plasmid DNA. Alternative vectors may include the bacterial promoter and selectable marker as a separate unit from the eukaryotic marker. Cells containing vector and flanking genomic DNA are selected for expression of the Neo activity within TKβGEO by growth on kanamycin containing plates. Plasmid DNA is then recovered from kanamycin resistant colonies and used in sequencing reactions to determine the polynucleotide sequence of flanking genomic DNA. Following sequencing, the identity of the gene and the location of the vector insertion site into the human genome are determined by comparison to sequences found in the human genome database, such as the NCBI database

F) Confirmation of Intron Insertion Site and Recombinant mRNA Identity by Sequencing of RT-PCR Fragments

The predicted recombinant mRNA sequences are ascertained by identifying the integration site of the integration vector, such as an intron of a gene. The predicted recombinant mRNA sequences may contain exon sequences that reside 5′ to the insertion site and vector sequences that reside 3′ to the SA and 5′ to the poly-A site of the vector. Sequencing of the recombinant RNA can confirm the prediction. For example, the sequence of recombinant mRNAs may be determined by using reverse transcriptase to generate a cDNA product using primers homologous to sequences residing in vector-derived mRNA. The cDNA is then used with PCR to amplify recombinant cDNA sequences, using primers homologous to vector-derived sequences and primers directed at predicted exon sequences, such as those near the 5′ end of the predicted mRNA. PCR products are sequenced to determine exon utilization in the recombinant mRNA. Methods for RT-PCR and sequencing of PCR products are well known in the art.

G) Gene-Specific, RNAi Suppression of TKβGal Expression

One aspect of the invention is the ability to suppress expression of gene trap selectable markers and reporters through use of gene-specific selection molecules. Gene trapped cells express recombinant mRNAs comprised of gene-derived exons that reside 5′ to the integration site and vector sequences that reside 3′ to the SA and 5′ to the poly-A site. These include GTX and TKβGEO, which expresses Neo for positive selection on G418, β-galactosidase for reporter functionality, and TK for negative selection on gancyclovir.

Expression of recombinant mRNAs is suppressed through use of selection molecules, including siRNA and vectors for expression of self-annealing, hairpin RNAs that result in RNAi.

An selection molecule is directed to exon-derived sequences (the recognition site for a selection molecule) contained in the recombinant mRNA. RNAi directed cleavage of mRNA leads to degradation of entire mRNAs (Javorschi, S. et. al., PharmaGenomics, February, 2004.). Hence, cells treated with selection molecules directed to exon sequences are selected by resistance to gancyclovir or by suppression of β-galactosidase activity. In this example, RNAi suppression of TKβGEO is target gene specific, in that the RNAi does not recognize every other cell containing an integrated vector. However, in addition to suppressing expression of the recombinant mRNA, the RNAi molecule suppresses expression of wild type mRNAs for the targeted gene that derive from additional copies of the gene residing in the genome.

Rather than directing RNAi molecules to gene-derived sequences alone, RNAi may be directed to a hybrid recognition site in the recombinant RNA that is derived from genomic sequences and vector sequences. In this example, the primary gene transcript is processed by the splicing machinery of the cell to generate a recombinant mRNA comprised of endogenous exon sequences located 5′ to the SA and vector sequences residing 3′ to the SA and 5′ to the poly-A site. Hence, the splicing machinery of the cell creates a unique hybrid recognition site at the junction of exon-derived and vector-derived mRNA. In this case, sequences residing 5′ to the splice donor represent the the 5′ portion of the hybrid recognition site (the 5′ hybrid recognition site) while vector sequences residing 3′ to the SA represent the 3′ portion of the hybrid recognition site (the 3′ hybrid recognition site). The 5′ hybrid recognition site combines with the 3′ hybrid recognition site to reconstitute the complete hybrid recognition site. In random integration vectors, the efficiency of the RNAi reaction can be controlled by selection of the number of nucleotides comprising the 3′ hybrid recognition site and the composition of the nucleotides. For example, the 3′ hybrid recognition site may be comprised of 15 out of 19 nucleotides of the sense strand, and 13 out of 19 nucleotides of the antisense strand of the hybrid recognition site. The sense strand sequence may further conform to algorithms, such as the Reynolds algorithm, thereby ensuring high probability for effective RNAi.

Following introduction of a hybrid RNAi, effects on β-galactosidase activity are monitored. By using algorithms, such as the Reynolds algorithm, to identify or design hybrid recognition sites, suppression of 50% to 79%, more preferably 80% to 90%, and most preferably greater than 90% of of β-galactosidase activity is attained.

Cells are treated with siRNA reagents and selected for suppression of mRNA expression by growth on gancyclovir. Viability is determined by a vital dye indicator (e.g., Alamar Blue assay) and plating efficiency is determined by counting surviving colonies growing on the gancyclovir containing plates). Controls are cells treated with non-selective siRNA reagents (e.g., randomized siRNA sequence such that no gene in the genome is recognized, or siRNA directed to a foreign gene such as to green florescent protein) for comparison.

### Example 2

### Selection of Cells with Vector DNA Integrated into Genes of Interest; 5′ Gene Trap Elements

An integration vector comprised of the following components is introduced into a mammalian (human) cell: from 5′ to 3′, a flip recombinase target sequence (FRT), a SA, a 3′ hybrid recognition site, translation stop codons in all 3 reading frames (STOP), EM-7, GTX, TKβGEO, a PA site, ORI, and a second FRT sequence. If using a plasmid based vector, the vector is typically linearized by restriction digestion prior to introduction into cells. Upon integration into an intron, recombinant primary transcripts comprised of genome-derived RNA plus FRT, SA, the hybrid recognition site, STOP, EM-7, GTX, TKβGEO, with a poly A tail are produced. The recombinant primary gene transcripts are processed by the splicing machinery of the cell to generate cells with different recombinant mRNAs comprised of endogenous exon sequences located 5′ to the SA and vector sequences residing 3′ to the SA and 5′ to the poly-A tail. Included in the mRNA are the hybrid recognition site, STOP, EM-7, GTX, and TKβGEO components. The endogenous promoter of the trapped gene drives expression of these mRNAs in which translation of exon-derived mRNA terminates at the vector derived STOP sites and in which ribosomes initiate translation from the GTX IRES to express the TKβGEO fusion gene. Such gene trapped cells are resistant to G418.

Selection of cells in which plasmid sequence has integrated into transcriptionally active DNA is accordingly accomplished by selection on G418. To identify cells with vector sequences integrated into a desired gene, such as into an intron of the desired gene, RNAi molecules are introduced into G418 resistant cells. In one case, the RNAi molecules are designed against recognition sites within the recombinant RNA that are derived from genomic DNA. An algorithm, such as the Reynolds algorithm, is used to identify effective recognition sites. Following introduction into the human cells, the RNAi molecule suppresses expression of the mRNAs to which it was designed, but not mRNAs to which it was not designed. Accordingly, both the wild type mRNA for the gene and the recombinant mRNA for the gene are silenced. Such cells with suppressed expression of TKβGEO are selected either by selection on gancyclovir or by monitoring β-galactosidase activity by FACS analysis, for example. The resulting population contains cells highly enriched for cells with vector integrated into the gene of interest. To confirm integration into such genes and to confirm the integration site, technologies such as Northern blots, Southern blots, and gene-tagging/plasmid rescue and sequencing may be employed.

Alternatively, one or more hybrid RNAi molecules may be designed specifically for the gene of interest. For example, the 3′ sequences for many gene exons are known and available from genome data bases. To arrive at a hybrid RNAi molecule, a 5′ hybrid recognition site is identified in one or more exons of a gene of interest. With this information, a corresponding 3′ hybrid recognition site is designed using algorithms, such as the Reynolds algorithm. Design elements include a) the number of nucleotides contained in the 3′ hybrid recognition site and 2) the nucleotide sequence. With this ability to optimize hybrid recognition sites, highly effective RNAi molecules are obtained. The optimal RNAi molecules are then introduced into G418 resistant cells and screened using gancyclovir or β-galactosidase and confirmed using conditions described in the preceding paragraph.

### Example 3

### Selection of Cells with Vector DNA Integrated into Genes of Interest; 3′ Gene Trap Elements

An integration vector comprised of the following components is introduced into a mammalian (human) cell: From 5′ to 3′, a first FRT, STOP, a cytomegalovirus promoter/enhancer (CMV), EM-7, TKβGEO, a 5′ hybrid recognition site, a SD, ORI, and a second FRT sequence.

If using a plasmid based vector, the vector is typically linearized by restriction digestion prior to introduction into cells. Upon integration into introns, primary transcripts comprised of the TKβGEO, the hybrid recognition site, SD, plus genomic DNA terminated at a PA are produced. The recombinant primary gene transcripts are processed by the splicing machinery of the cell to generate cells with different recombinant mRNAs comprised of TKβGEO, the hybrid recognition site, plus endogenous exon sequences located 3′ to the integration site of the vector, terminated by polyadenylation. The CMV promoter of the vector drives expression of these to express the TKβGEO fusion gene. Such gene trapped cells are resistant to G418. Selection of cells in which vector sequence have integrated into transcriptionally active DNA is accordingly accomplished by selection on G418.

To identify cells with vector sequences integrated into a desired gene, such as into an intron of the desired gene, RNAi molecules are introduced into G418 resistant cells. In one case, the RNAi molecules are designed against RNAi recognition sequences within the recombinant RNA that are derived from genomic DNA. An algorithm, such as the Reynolds algorithm, is used to identify effective RNAi recognition sequences. Following introduction into the human cells, the RNAi molecule suppresses expression of the mRNAs to which it was designed, but not mRNAs to which it was not designed. Accordingly, both the wild type mRNA for the gene and the recombinant mRNA for the gene are silenced. Such cells with suppressed expression of TKβGEO are selected either by selection on gancyclovir or by monitoring β-galactosidase activity by FACS analysis, for example. The resulting population contains cells highly enriched for cells with vector integrated into the gene of interest. To confirm integration into such genes and to confirm the integration site, technologies such as Northern blots, Southern blots, and gene-tagging/plasmid rescue and sequencing may be employed.

Alternatively, one or more hybrid recognition sites may be designed specifically for the gene of interest. For example, the 5′ sequences for many gene exons are known and available from genome data bases. To arrive at a hybrid recognition site, a 3′ hybrid recognition site is identified in one or more exons of a gene of interest. A corresponding 5′ hybrid recognition site is designed using algorithms, such as the Reynolds algorithm. Design elements include a) the number of nucleotides contained in the 5′ hybrid recognition site and 2) the nucleotide sequence. With this ability to optimize hybrid recognition sites, highly effective selection molecules are obtained. The optimal RNAi molecules are then introduced into G418 resistant cells and screened using gancyclovir or β-galactosidase and confirmed using conditions described in the preceding paragraph.

### Example 4

### Homologous Recombination Integration Vectors with 5′ or 3′ Gene Trap Elements

Gene trapping vectors from examples 2 and 3 may further comprise homology domains at one or both ends of the integration vector. Homology domains serve to increase the frequency of integration into desired genetic loci, such as genes, and they serve to direct integration to a specific site within a gene (e.g. an intron). This ability to pin-point the integration vector within specific nucleotides is not readily accomplished with randomly integrating vectors. By increasing the frequency of integration into a desired locus, the number of cells required to screen for cells with vector integrated into desired genetic loci or genes is also reduced.

As example, a homologous recombination integration vector comprised of the following components is introduced into a mammalian (human) cell: From 5′ to 3′, first homology domain (HD1), a first FRT, a SA, a 3′ hybrid recognition site, STOP, EM-7, GTX, TKβGEO, PA, ORI, a second FRT sequence, and a second homology domain (HD2), in which HD1 is 5′ to HD2 in genomic DNA and in which HD1 and HD2 adjoin one another in genomic DNA. If using a plasmid based vector, the vector is typically linearized by restriction digestion prior to introduction into cells. Following introduction into mammalian (human) cells, selection for gene-specific integration is as described for Example 2.

As example, a homologous recombination integration vector comprised of the following components is introduced into a mammalian (human) cell: From 5′ to 3′, HD1, a first FRT, STOP, CMV, EM-7, TKβGEO, a 5′ hybrid recognition site, SD, ORI, a second FRT sequence, and HD2, in which HD1 is 5′ to HD2 in genomic DNA and in which HD1 and HD2 adjoin one another in genomic DNA. If using a plasmid based vector, the vector is typically linearized by restriction digestion prior to introduction into cells. Following introduction into mammalian (human) cells, selection for gene-specific integration is as described for Example 3.

### Example 5

### Converting Vectors: e.g. from a 3′ Gene Trap Core to a 5′ Gene Trap Core

Recombinase sites, such as FRT sites, are used to manipulate vector sequences that reside between them. In some cases, it is desirable to exchange one genetic sequence with another. For example, it is often desirable to exchange a wild-type allele with a mutant allele, or a mutant allele with a wild type allele. To exchange one exon of a gene with an alternative, mutant exon (e.g. a mutant or splice variant), a vector comprising the following elements are introduced into host cells: from 5′ to 3′, HD1, a first FRT, SA, a 3′ hybrid recognition site, GTX, EM-7, TKβGEO, a PA, a second FRT site whose 8 nucleotide core is oriented in the same direction as the first FRT site and which is capable of recombination with said first FRT site (FRT), a second SA, a mutant exon, a SD, and ORI, wherein HD1 is 5′ to HD2 in genomic DNA and wherein HD1 is 5′ to the exon to be replaced and wherein HD2 is 3′ to the exon to be replaced. If using a plasmid based vector, the vector is linearized by restriction digestion prior to introduction into cells. Selection of cells with vector integrated into the desired locus is performed as described for Example 2.

Flp recombinase is then introduced into the host cells by methods known in the art, such as introduction of purified protein or an expression vector. The Flp recombinase catalyzes excision of vector sequences residing between the FRT sites, leaving one copy of the FRT site intact. Removal includes the first SA, the 3′ hybrid recognition site, GTX, the EM-7 promoter, TKβGEO, and the PA. Cells with excised vector sequences are accordingly selected by resistance to gancyclovir or by suppression of β-galactosidase activity. Through this process, the exon originally in the genomic DNA is replaced by the exon contained in the homologous recombination vector. The newly integrated exon is now expressed and processed into the host gene product.

### Example 6

### Creation of β-Galactosidase Reporter Cells for HOXB13 and IL17BR. in MCF-7 Cells by Homologous Recombination

The ratio of HOXB13 (which is over-expressed in breast cancer cells) and IL17BR (which is under-expressed in breast cancer cells) accurately predicts tumor relapse following adjuvant tamoxifen monotherapy. The signaling pathways regulating expression of these genes are therefore abnormal in patients susceptible to relapse, as compared to patients that do not relapse. Accordingly, drug products that target these pathways would be expected to have therapeutic benefit, by suppressing relapse of breast cancer patients. Using the methods and vectors described above, gene expression reporters for HOXB13 and IL17BR are produced in a breast cancer cell line as follows. The cells are probed with siRNA reagents and chemical entities to identify regulatory genes, pathways and drug candidates.

The core 5′ gene trap vector utilized herein is comprised of the following components: From 5′ to 3′, SpeI, HindIII, AvrII, and NsiI restriction sites, a first FRT, a SA, a 3′ hybrid selection site, STOP, EM-7, GTX, TKβGEO, a PA, an ORI, and a second FRT sequence.

The HOXB13 gene resides at chromosome 17, and codes for a 1.27 KB mRNA (GenBank accession # NM_{—}006361). To target gene trap elements to the first intron of HOXB13, the nucleotide sequence for the first intron of the gene is determined through use of the human genome database. A homology domain of ˜900 bp within the first intron is generated by PCR from human genomic DNA with restriction sites HindIII and BstBI incorporated into the forward primer, and AvrII site into the reversed primer. The fragment is cleaved with HindIII and AvrII and ligated into the HindIII and AvrII sites in the vector. Similarly, a second 2 KB homology domain is synthesized using sequence data immediately 3′ to that for the first homology domain. Forward primers include SpeI while the reverse primer incorporates BstBI. This sequence is introduced into the unique SpeI and BstBI sites of the vector after prior insertion of the first homology domain. Vectors are amplified in bacteria in the presence of Kanamycin, which selects for cells expressing Neo within TKβGEO from the EM-7 promoter. The design of the vector is such that when the plasmid DNA is linearized with BstBI, the first homology domain resides 5′ to the SA site in the pGTX-TKβGEO vector while the second homology domain resides 3′ to the site.

The IL17BR gene resides at chromosome 3, and codes for a 2 KB mRNA (GenBank accession # AY518533). To target gene trap elements to the first intron of IL17BR, the nucleotide genomic sequence for the first intron of the gene was determined through use of the human genome database. A 1.9 KB homology domain within the first intron is generated by PCR from human genomic DNA with restriction sites HindIII and BstBI incorporated into the forward primer, and NsiI site into the reversed primer. The resulting fragment is cleaved with HindIII and NsiI and ligated into the HindIII and NsiI sites in the vector. Similarly, a second 2 KB homology domain is synthesized with restriction site XbaI incorporated into the forward primer, and BstBi site into the reversed primer using sequence data immediately 3′ to that for the first homology domain. This sequence will be introduced into the unique SpeI and BstBI sites of the vector after prior insertion of the 1^{st} homology domain. Vectors are amplified in bacteria in the presence of Kanamycin, which selects for cells expressing Neo within TKβGEO from the EM-7 promoter. The design of the vector is such that when the plasmid DNA is linearized with BstBI, the first homology domain resides 5′ to the SA site in pGTX-TKβGEO while the second homology domain resides 3′ to the PA site.

The HOXB13 homologous integration vector is introduced into MCF-7 cells by electroporation. The IL17BR homologous integration vectors is introduced into a different population of these cells. Cells with vectors integrated into introns of genes, either by homologous recombination or through random integration, use the “trapped” endogenous promoters to drive expression of TKβGEO. They are selected by growth on G418.

RNAi molecules (e.g. siRNAs) are then used to suppress expression of TKβGEO. The siRNA reagents are designed for optimal activity using algorithms, such as the Reynolds algorithm. Four sets of siRNA reagents are utilized. As a control for transfection efficiency, the first set utilizes siRNA reagents directed against TK. When introduced into host cells, this siRNA reagent will suppress expression of TKβGEO expression in all cells, whether integrated by homologous recombination or by random integration. Such cells have reduced β-galactosidase activity and are resistant to gancyclovir. As a control for non-specific effects of transfection, the second set utilizes a randomized siRNA sequence that has no corresponding sequence in the human genome. When introduced into host cells, this siRNA reagent is not expected to suppress TKβGEO expression. Such cells maintain β-galactosidase activity and remain sensitive to gancyclovir.

To select for cells integrating into the human genome by homologous recombination, the third set includes siRNA reagents directed at sequences contained in the first exon of either HOXB13 or IL17BR. Following homologous recombination, but not in randomly integrated cases, mRNA derived from the first exon of HOXB13 or IL17BR is linked in a recombinant mRNA with TKβGEO sequences. Therefore, when introduced into host cells, the siRNA reagents are expected to preferentially suppress TKβGEO in cells that have integrated into the human genome by homologous recombination, but not cells integrating vector randomly into the genome. Such cells have reduced β-galactosidase activity and are resistant to gancyclovir. However, it is of note that the recombinant mRNA and the wild type HOXB13 or IL17BR mRNA expressed from other copies of these genes in the genome will be suppressed by these siRNA reagents. Accordingly, if suppression of HOXB13 or IL17BR is lethal to cells, all cells (both homologously integrated and randomly integrated) would be killed, even without gancyclovir selection. Moreover, in some small percentage of cells, suppression of the endogenous HOXB13 or IL17BR gene regulates expression of other genes into which some vectors have integrated. Accordingly, these cells with randomly integrated vector also exhibit suppressed β-galactosidase activity and are resistant to gancyclovir. The population is significantly enriched for cells with homologously integrated vector.

As a preferred method for selecting for cells integrating vector into the human genome by homologous recombination, a fourth set includes siRNA directed at the junction of genome-derived and vector derived sequences. The siRNAs are designed using some of the rules for effective gene silencing (e.g. the Reynolds algorithm). This includes the last nine nucleotides of exon1 (sense strand) of HOXB13 (e.g. CAUUUGCAG) and the first 10 nucleotides (sense strand) following the SA in the gene trap vector (UACUGUAUAA) or the last nine nucleotides of exon 1 (sense strand) of IL17BR (e.g. CGAGAGCCG) and the first 10 nucleotides following the SA (sense strand) in the gene trap vector (e.g. UAAUGUAUAA). The sense strand of the RNAi molecule for HOXB13 is therefore CAUUUGCAGUACUGUAUAA (5′ to 3′) while the antisense strand is UCGUAAACGUCAUGACAUA (3′ TO 5′), which has a Reynolds score of 9 out of 10. The sense strand of the RNAi molecule for IL17BR is therefore CGAGAGCCGUAAUGUAUAA (5′ to 3′) while the antisense strand is GGGCUCUCGGCAUUACAUA (3′ TO 5′), which has a Reynolds score of 10 out of 10. Since this sequence is only found in the chimeric mRNA, complications due to suppression of the endogenous wild type gene are eliminated. Accordingly, when introduced into recipient cells, this siRNA reagent suppresses only TKβGEO in cells that have integrated into the human genome by homologous recombination. Such cells have suppressed β-galactosidase activity and are resistant to gancyclovir.

Following selection and cloning of cells selected for suppressed β-galactosidase activity or resistance to gancyclovir, validation of integration site is performed using 3 methods:

First, PCR primers are used to amplify genomic DNA sequences containing vector, homology domain 1 also contained in the homologous recombination vector, and flanking genomic DNA. Nested primers within vector DNA (3′ to the SA) and within flanking genomic DNA of the target site are used. Accordingly, PCR fragments are only obtained from cells with vector sequences integrated by homologous recombination (e.g. PCR-positive cells). Confirmation of flanking genomic DNA is performed by sequencing, using primers within the vector and within the homology domain.

Second, genomic DNA is obtained from PCR-positive cells and cut with, for instance, SacII restriction endonuclease. This endonuclease cuts ˜300 base pairs 5′ to the 5′ homology domain of HOXB13, ˜1500 nucleotides 3′ to the 5′ homology domain, and once at ˜2500 bp from the SA site in the vector within the lacZ coding region. The wild type sequence is 2700 bp, while the homologous recombinant fragment is 3700 bp. Probe for Southern blotting will be derived from PCR products of the wild type DNA spanning the coding sequence of exon 1 of HOXB13. In wild type cells or cells with vector integrated randomly into the genome, this probe reveals only the wild type fragment on Southern blots at 2.7 KB. For cells with homologously integrated DNA, band at 3.7 KB representing the homologous recombinant fragment will be identified.

Third, a gene-tagging/plasmid rescue method is utilized. With this method for the case of HOXB13 targeting, genomic DNA from PCR-positive cells are cut with AvrII restriction endonuclease. This endonuclease cuts once within the vector, 5′ to the EM-7 bacterial promoter and 3 KB 3′ to the 3′ homology domain. The restriction digested genomic DNA is then self-ligated and used to transform bacteria. Included in the transformed bacteria are cells containing recombinant DNA including the EM-7 bacterial promoter driving expression of TKβGEO, a ORI, the 3′ homology domain, and 3′ flanking genomic DNA. Amplification and selection for these cells is accomplished by growth on Kanamycin (selection for expression of NEO within the TKβGEO gene). Sequencing primers upstream of TK are then used to sequence from vector DNA into the adjoining 3′ flanking genomic DNA. The sequence is then aligned with the human genome database to confirm the predicted sequence of the flanking genomic DNA and, hence, homologous recombination.

Both Southern blots and gene tagging/plasmid rescue methods do not use PCR to amplify recombinant DNA and hence avoid any PCR artifacts that may occur. Of these, gene tagging/plasmid is a reliable means for validation.

### Example 7

### Discovery of Pathways and Genes Regulating Expression of HOXB13 and IL17BR by Genetic Screening with RNAi and Chemical Agents

To discover pathways and genes regulating HOXB13 and IL17BR, the gene expression reporter cells produced in Example 6 are screened with select siRNA reagents and chemical reagents with known targets and mechanism of action. The reporter gene, β-galactosidase, is regulated by either HOXB13 or IL17BR promoters. By residing at the normal chromosomal locus, the effect of regulatory sequences residing close to the promoter for these genes as well as those residing long distances from the promoter (e.g. as much as 50 KB away) are reflected in expression of β-galactosidase activity. β-galactosidase is chosen as reporter, due to the ease of performing high throughput (automated) assays using cell extracts or using live cells. β-galactosidase is also applicable to flow cytometry methods, to sort for cells with expressed or suppressed expression.

To identify genes and pathways regulating expression of HOXB13 and IL17BR, a library of siRNA reagents are purchased from commercial supplier.

To control for transfection efficiency and non-specific effects due to transfection procedures, the siRNA library includes those directed at TK and randomized nucleotides with no corresponding sequence in the human genome. SiRNA reagents are introduced into cells in a 96-well format (triplicate samples). If a siRNA suppresses expression of a gene that regulates expression of HOXB13 or IL17BR, β-galactosidase activity in the respective reporter cells is modulated. If the targeted gene is a suppressor of HOXB13 or IL17BR, expression of the reporter increases relative to the randomized siRNA control. If the targeted gene is an inducer of HOXB13 or IL17BR, expression of the reporter decreases relative to the randomized siRNA control. If the targeted gene is not involved in regulation of HOXB13 or IL17BR, no change in expression of reporter is detected.

To distinguish between genes with general effects on transcription versus those that specifically regulate HOXB13 or IL17BR, siRNA reagents that demonstrate regulatory effects on HOXB13 or IL17BR are further evaluated for effects on transcription of additional reporter cells, generated by random incorporation of gene trap vectors into recipient cells. Specific regulators of HOXB13 or IL17BR do not have effects on β-galactosidase activity produced by these randomly generated cells, in contrast to non-specific regulators of gene expression that would. Genetic screening or reporter cells with siRNA reagents is highly efficient, requiring 1-2 weeks to perform multiple experiments.

In addition to genetic screening using siRNA reagents, libraries of chemical reagents with known targets and mechanisms of action are commercially available chemical diversity libraries for drug screening are also known in the art. Screening of chemical libraries with reporter cells is highly efficient, requiring 1-2 weeks to perform multiple experiments.

Screening with siRNA and chemical agents is not absolute, as non-specific effects of these reagents can be observed. Following preliminary studies with alternative siRNA reagents to targeted gene products provides additional credibility to demonstrated effects. Similarly, many of the targets represented in the library have multiple (redundant) chemical inhibitors. Consistent effects with redundant inhibitors verify the targeted gene product. Additional techniques, such as use of Northern blots and quantitative PCR are used to confirm results from screening of siRNA and chemical entities.

1. An integration vector for modifying a target genomic region comprising, in a 5′ to 3′ direction: a splice acceptor site, a 3′ hybrid recognition site, and a marker sequence.

2. An integration vector according to claim 1, further comprising a first recombinase target sequence 5′ to the marker sequence and a second recombinase target sequence 3′ to the marker sequence.

3. An integration vector according to claim 1, further comprising, 3′ to the marker sequence, a polyadenylation site or a splice donor site.

4. An integration vector according to claim 1, further comprising a bacterial origin of replication or a bacterial promoter operably linked to a bacterial selection marker or both.

5. An integration vector according to claim 1, further comprising an internal ribosome entry site between the 3′ hybrid recognition site and the marker sequence.

6. An integration vector according to claim 5, wherein the internal ribosome entry site comprises the 5′ leader sequence of the mRNA encoding GTX homeodomain protein.

7. An integration vector according to claim 5, further comprising one or more stop codons 5′ to the internal ribosome entry site.

8. An integration vector according to claim 1, wherein the marker sequence is a thymidine kinase, β-galacatosidase, neomycin resistance fusion gene (TKβGEO).

9. An integration vector according to claim 1, wherein the vector lacks internal sites for recognition by a frequently cutting restriction enzyme.

10. An integration vector according to claim 1, further comprising a first homologous domain 5′ to the marker sequence and a second homologous domain 3′ to the marker sequence, wherein the first and second homologous domains have substantial homology with first and second nucleic acid sequences of the target genomic region.

11. An integration vector according to claim 1, wherein the genome region comprises a cellular gene, and wherein said vector further comprises a sequence selected from the group consisting of a splice variant of the gene, a replacement for the gene, a mutant sequence of the gene, a SNP variant of the gene, and a promoter to express the gene.

12. An integration vector for modifying a genome region, comprising, in a 5′ to 3′ direction: a marker sequence, a 5′ hybrid recognition site, and a splice donor site.

13. An integration vector according to claim 12, further comprising a first recombinase target sequence 5′ to the marker sequence and a second recombinase target sequence 3′ to the marker sequence.

14. An integration vector according to claim 12, further comprising a bacterial origin of replication or a bacterial promoter operably linked to a bacterial selection marker or both.

15. An integration vector according to claim 12, wherein the marker sequence is a thymidine kinase, β-galacatosidase, neomycin resistance fusion gene (TKβGEO).

16. An integration vector according to claim 12, wherein the vector lacks internal sites for cutting by a frequently cutting restriction enzyme.

17. An integration vector according to claim 12, further comprising a first homologous domain 5′ to the marker sequence and a second homologous domain 3′ to the splice donor site, wherein the first and second homologous domains have substantial homology with first and second nucleic acid sequences of the target genomic region.

18. An integration vector according to claim 12, wherein the genome region comprises a cellular gene, and wherein said vector further comprises a sequence selected from the group consisting of a splice variant of the gene, a replacement for the gene, a mutant sequence of the gene, a SNP variant of the gene, and a promoter to express the gene.

19. An integration vector according to claim 12, further comprising a promoter that is operably linked to said marker sequence.

20. An integration vector for modifying a genome region, comprising, in a 5′ to 3′ direction: a splice acceptor site, a 3′ hybrid recognition site, an internal ribosome entry site, a marker sequence, a 5′ hybrid recognition site, and a splice donor.

21. An integration vector according to claim 20 further comprising a stop codon, which is located between the splice acceptor site and internal ribosome entry site, and a bacterial promoter operably linked to a bacterial selection marker, which are located between the splice acceptor site and the marker gene.
